(19) United States
(12) Patent Application Publication
(10) Pub. No.: US 2015/0133412 A1
(43) Pub. Date: May 14, 2015

(54) Methods for Detecting and Treating Rhinovirus Infection

(71) Applicant: The Procter & Gamble Company, Cincinnati, OH (US)

(72) Inventors: Robert Joseph Isfort, Fairfield, OH (US); Xiaoyan Angela QU, Cary, NC (US)

(73) Assignee: The Procter & Gamble Company

(21) Appl. No.: 14/571,429

(22) Filed: Dec. 16, 2014

Related U.S. Application Data
(63) Continuation of application No. 14/508,017, filed on Oct. 7, 2014.
Application No. 14/508,017 is a continuation of application No. 13/719,266, filed on Dec. 19, 2012.
(60) Provisional application No. 61/578,369, filed on Dec. 21, 2011.

(51) Int. Cl. C12Q 1/70; A61K 31/4402; A61K 31/573; A61K 31/485; A61K 31/137

(52) U.S. Cl. CPC C12Q 1/701; A61K 31/485; A61K 31/137; A61K 31/573; A61K 31/4402
USPC 514/171; 506/9; 514/289

(57) ABSTRACT

The invention provides a method for evaluating the activity of an agent for treating rhinovirus infection or a symptom thereof, a method of detecting or monitoring rhinovirus infection, and a method of treating rhinovirus infection or a symptom thereof. Various embodiments comprise measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample to produce a gene expression profile, and comparing the gene expression profile to a reference gene expression profile. Systems, computer readable media, compositions, and methods for maintaining or improving respiratory health also are provided.

### CROSS REFERENCE TO RELATED APPLICATIONS

This application is a continuation of U.S. application Ser. No. 14,508,017 filed on Dec. 19, 2012; which claimed the benefit U.S. Provisional Application No. 61/578,369 filed on Dec. 21, 2011.

### FIELD OF THE INVENTION

The invention relates to the use of gene expression profiles to determine the presence or severity of rhinovirus infection and compounds and methods for maintaining or improving respiratory health.

### BACKGROUND OF THE INVENTION

Rhinoviruses are small, non-enveloped plus-strand RNA-containing viruses belonging to the Picornaviridae family. The primary site of infection of a host is the nasal mucosa. Rhinovirus attaches to respiratory epithelium and spreads locally, traveling to the nasal pharynx. Upon infection of epithelial cells, viral replication begins and viral shedding occurs within 8-10 hours, with as many as 1 million infectious virions present per milliliter of nasal washing.

Rhinovirus is the most prevalent pathogen associated with acute exacerbations of asthma and chronic obstructive pulmonary disease (COPD), and rhinovirus infection accounts for approximately 30-50% of cases of the “common cold.” Common cold symptoms include sneezing, malaise, stuffy or runny nose, wheezing, sore throat, coughing, watery eyes, headache, body ache, ear ache or inflammation, sinusitis, and fever. It is estimated that adults suffer two to three colds per year, and children suffer five to seven colds per year. While most infections are associated with mild symptoms and last seven to ten days, some patients develop severe respiratory complications, such as pneumonia. The economic effect of colds also is not insignificant. Colds are responsible for 50% of short-term absences from work and school. Effective treatment to decrease symptom severity, shorten the duration, and decrease the incidence of colds has been an elusive goal. Similarly, the ability to distinguish rhinovirus-based respiratory symptoms from other respiratory ailments can be challenging, which hampers effective treatment of a patient.

There is a continuing need for methods of identifying and monitoring rhinovirus infection, as well as screening methods for identifying new therapeutic targets for treating infection and symptoms associated therewith.

### SUMMARY OF THE INVENTION

The invention provides materials, methods, and systems that will improve healthcare related to rhinovirus infection and respiratory health in general. For example, various aspects of the invention relate to methods of using a gene expression profile to evaluate a rhinovirus infection in a human patient and methods of characterizing the activity of an agent or effectiveness of a therapeutic regimen against rhinovirus infection.

In one aspect, the invention provides a method for evaluating the activity of an agent for treating rhinovirus infection or a symptom thereof. The method comprises administering an agent to an animal infected with rhinovirus, and measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample from the animal to produce a gene expression profile. A gene expression profile that differs from a reference gene expression profile indicates that the agent treats rhinovirus infection or a symptom thereof. In various embodiments, the reference gene expression profile is a reference gene expression profile of infection, and (i) an increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or (ii) a decrease in expression of FAS, PLSCR1, CLEC2B, BATF3, HAS2, MX1, SP110, GBP1, IFIT3, IFIT1, CXCL9, CXCL10, and/or CXCL11 compared to the reference gene expression profile of infection indicates that the agent treats rhinovirus infection or a symptom thereof.

The invention also includes a method of detecting or monitoring rhinovirus infection in a human patient. The method comprises measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample from a human patient to produce a gene expression profile. The method further comprises comparing the gene expression profile from the patient to a reference gene expression profile. A gene expression profile that (1) differs from a reference gene expression profile of non-infection or (2) does not significantly differ from a reference gene expression profile of infection is indicative of rhinovirus infection. A gene expression profile having an increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or (2) a decrease in expression of FAS, PLSCR1, CLEC2B, BATF3, HAS2, MX1, SP110, GBP1, IFIT3, IFIT1, CXCL9, CXCL10, and/or CXCL11 compared to the reference gene expression profile of infection is indicative of improvement or amelioration rhinovirus infection or symptoms thereof; an undetectable or insignificant difference(s) is indicative of infection or failure to respond to treatment. Alternatively or in addition, the gene expression profile is compared with a reference gene expression profile of non-infection. In this regard, a gene expression profile having a decrease in expression of RNFT2, CRY2, C10ORF95, BTG4, PSD3, CAPN9, SULT1E1, HEY1, LRRC36, RAB3B, ALDH3B1, FAM134B, and/or B3GAT3, and/or (2) an increase in expression of FAS, PLSCR1, CLEC2B, BATF3, HAS2, MX1, SP110, GBP1, IFIT3, IFIT1, CXCL9, CXCL10, and/or CXCL11 compared to the reference gene expression profile of non-infection is indicative of rhinovirus infection.

The invention further provides a method of treating rhinovirus infection or a symptom thereof. The method comprises administering to a human patient an agent according to a therapeutic regimen for treating rhinovirus infection or a symptom thereof, and measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample from the patient to generate a gene expression profile. The method further comprises comparing the gene expression profile from the patient to a reference gene expression profile (e.g., a reference gene expression profile of infection or a reference gene expression profile of non-infection); and generating a new therapeutic regimen for the patient to treat rhinovirus infection or a symptom thereof if the gene expression profile (i) differs from a reference gene expression profile of non-infection or (ii) does not significantly differ from a reference gene expression profile of infection. In various embodiments, the method comprises generating a new therapeutic regimen if the gene expression profile has a decrease in expression of RNFT2, CRY2, C10ORF95, BTG4, PSD3, CAPN9, SULT1E1, HEY1, LRRC36, RAB3B, ALDH3B1, FAM134B, and/or B3GAT3, and/or an increase in expression of FAS, PLSCR1, CLEC2B, BATF3, HAS2, MX1, SP110, GBP1, IFIT3, IFIT1, CXCL9, CXCL10, and/or CXCL11, compared to a reference gene expression profile of non-infection. Alternatively, the method comprises generating a new therapeutic regimen if the gene expression profile has an undetectable or an insignificant increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or an undetectable or an insignificant decrease in expression of FAS, PLSCR1, CLEC2B, BATF3, HAS2, MX1, SP110, GBP1, IFIT3, IFIT1, CXCL9, CXCL10, and/or CXCL11, compared to a reference gene expression profile of infection.

A kit also is provided which comprises reagents for measuring expression of a gene panel packaged together. The gene panel consists of 2 to 10,000 genes and comprises (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. In any of the aspects of the invention, measuring gene expression in a biological sample is inclusive of, e.g., determining an amount of a polypeptide encoded by the gene(s) and/or an amount of an mRNA encoded by the gene(s) either by direct or indirect (e.g., by measure of a complementary DNA (cDNA) synthesized from the mRNA) measure of the mRNA.

A diagnostic test system is provided comprising a data collection tool adapted to collect data representative of expression measurements of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. The test system further comprises an analysis tool comprising a statistical analysis engine adapted to generate a representation of a correlation between the presence or severity of rhinovirus infection and expression measurements of the genes, wherein the representation of the correlation is adapted to be executed to generate a result; and an index computation tool adapted to analyze the result to determine the presence or severity of rhinovirus infection and represent the result as a numerical probability or a grade or score. The invention also includes a computer readable medium having computer executable instructions for determining the presence or severity of rhinovirus infection. The computer readable medium comprises a routine, stored on the computer readable medium and adapted to be executed by a processor, to store expression measurement data representing expression measurements of a gene panel; and a routine, stored on the computer readable medium and adapted to be executed by a processor, to analyze the expression measurement data to evaluate the presence or severity of rhinovirus infection, wherein the gene panel comprises (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11.

Also included in the invention is a method of formulating a composition for treating rhinovirus infection or a symptom thereof. The method comprises (a) accessing a plurality of instances stored on at least one computer readable medium, wherein each instance is associated with an agent and wherein each instance comprises an ordered list comprising identifiers representing gene expression transcripts encoded by (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. The method further comprises (b) comparing a rhinovirus infection-associated gene expression profile to the plurality of instances, wherein the rhinovirus infection-associated gene expression profile comprises identifiers representing gene expression transcripts encoded by (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, and wherein the comparison comprises comparing each identifier in the rhinovirus infection-associated gene expression signature with the position of the same identifier in the ordered lists for each of the plurality of instances. The method further comprises (c) assigning a connectivity score to each of the plurality of instances; and (d) formulating a composition comprising an agent associated with an instance having a negative connectivity score.

The invention includes, in one aspect, a composition comprising (i) a therapeutic agent selected from the group consisting of an antihistamine, an antitussive, a decongestant, an expectorant, and combinations thereof, and (ii) an excipient selected from the group consisting of olivem 450, usnic acid, silymarin, gum rosin, trideceth-10, and combinations thereof. A method of maintaining or improving respiratory health also is provided. The method comprises administering to a subject a composition comprising an excipient selected from the group consisting of olivem 450, usnic acid, silymarin, gum rosin, or trideceth-10. The use of an excipient selected from the group consisting of olivem 450, usnic acid, silymarin, gum rosin, or trideceth-10 in any of the methods disclosed herein or for preparation of medicaments for administration according to any of the methods disclosed herein is specifically contemplated. In this regard, the invention includes an excipient selected from the group consisting of olivem 450, usnic acid, silymarin, gum rosin, or trideceth-10 for use in a method of maintaining or improving respiratory health. Use of an excipient selected from the group consisting of olivem 450, usnic acid, silymarin, gum rosin, or trideceth-10 in the preparation of a medicament for maintaining or improving respiratory health also is provided.

### BRIEF DESCRIPTION OF THE DRAWINGS

While the specification concludes with claims particularly pointing out and distinctly claiming the subject matter that is regarded as the invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a table listing upregulated or downregulated gene transcripts in humans suffering from rhinovirus infection. The table provides the gene name (acronym), Entrez Gene identifier, the Affymetrix identifier corresponding to the probe that binds the gene transcript, and the “NetAffx Title” annotation describing the protein encoded by the gene transcript.

FIG. 2 is a schematic illustration of an exemplary system for producing a gene expression profile and comparing the gene expression profile to a reference gene expression profile.

FIG. 3 is a schematic illustration of a computer system suitable for use with the invention.

FIG. 4 is a schematic illustration of an instance associated with a computer readable medium, such as a computer readable medium of FIG. 3.

FIG. 5 is a schematic illustration of a comparison between a gene expression profile and an instance, wherein there is a positive correlation between the identifier lists.

FIG. 6 is a schematic illustration of a comparison between a gene expression profile and an instance, wherein there is a negative correlation between the identifier lists.

FIG. 7 is a schematic illustration of a comparison between a gene expression profile and an instance, wherein there is a neutral correlation between the identifier lists.

FIGS. 8A-8D are tables correlating the levels of IL-6, EP-10, and RANTES (pg/ml) observed three days following rhinoviral infection of BEAS-2B cells exposed to various doses of kaempferol, luteolin, quercetin, scutellarein, all-transretinoic acid, 3-hydroxyflavone, fustin, 3-methylquercetin, fisetin, apigenin, oxymetazoline HCl, chrysin, naringenin, pyrrolidinedithiocarbamate ammonium, 7-hydroxyflavone, myricetin, diosmetin, 4′,7-dihydroxyflavone, or eriodictyol. All assays were performed in triplicate. “Std” refers to standard deviation.

FIG. 9 is a table correlating the levels of IL-6, EP-10, and RANTES (pg/ml) observed two days following rhinoviral infection of BEAS-2B cells exposed to various doses of all-transretinoic acid, 3-methylquercetin, quercetin, or fisetin in combination with ibuprofen (20 M) (“+ibuprofen”) or without ibuprofen (“-ibuprofen”). All assays were performed in triplicate. “Std” refers to “standard deviation,” and “ave” refers to “average.”

FIG. 10 is a flow diagram of an example method for using a model to evaluate a subject for the presence or severity of rhinovirus infection.

### DETAILED DESCRIPTION OF THE INVENTION

The invention provides methods, systems, and tools which utilize a gene expression profile indicative of rhinovirus infection to, e.g., determine the presence or severity of rhinovirus infection, monitor patients undergoing a therapeutic regimen and alter the regimen to enhance efficacy, and evaluate the activity of potential therapeutics against rhinovirus infection or associated symptoms. The following text sets forth a broad description of numerous different embodiments of the invention. The description is to be construed as exemplary only and does not describe every possible embodiment since describing every possible embodiment would be impractical, if not impossible. It will be understood that any feature, characteristic, component, composition, ingredient, product, step, or methodology described herein can be deleted, combined with, or substituted for, in whole or part, any other feature, characteristic, component, composition, ingredient, product, step, or methodology described herein. Numerous alternative embodiments could be implemented, using either current technology or technology developed after the filing date, which would still fall within the scope of the claims. All publications and patents cited herein are incorporated herein by reference. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

### Gene Panels and Gene Expression Profiles

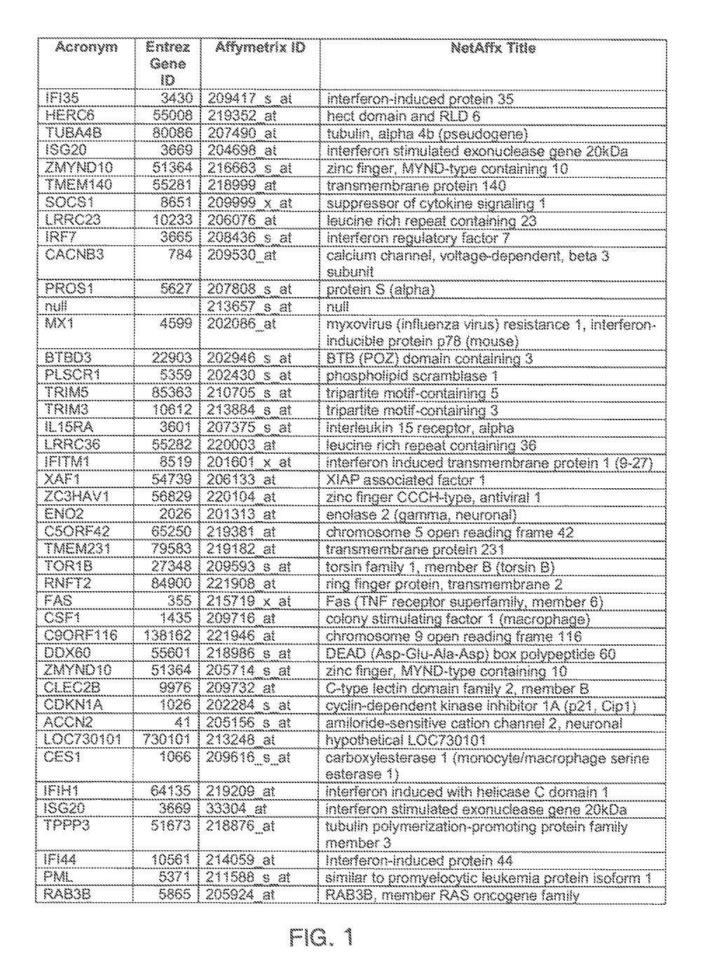

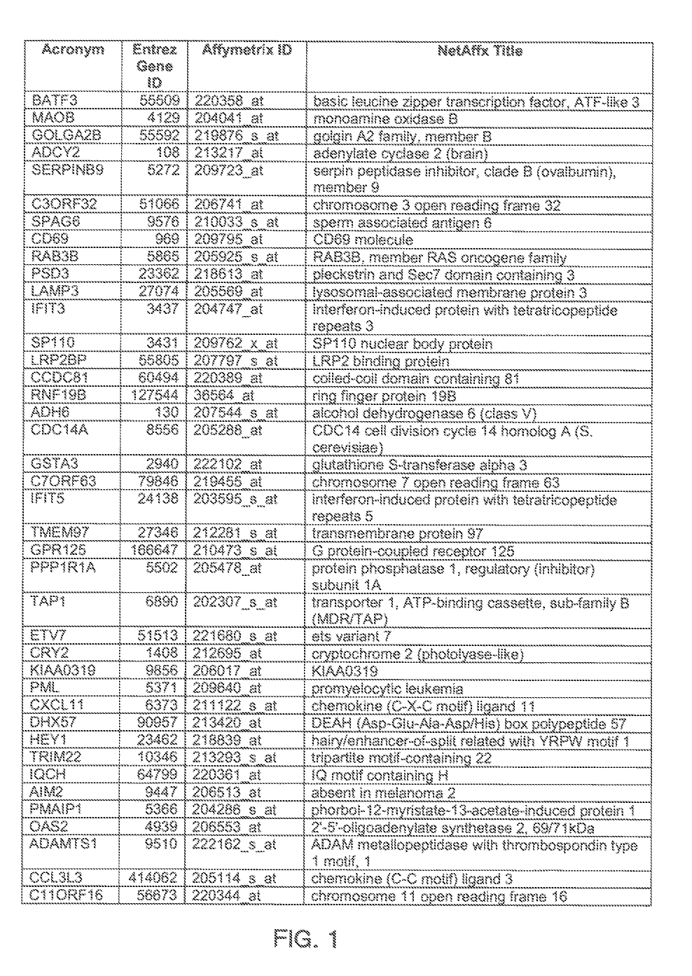

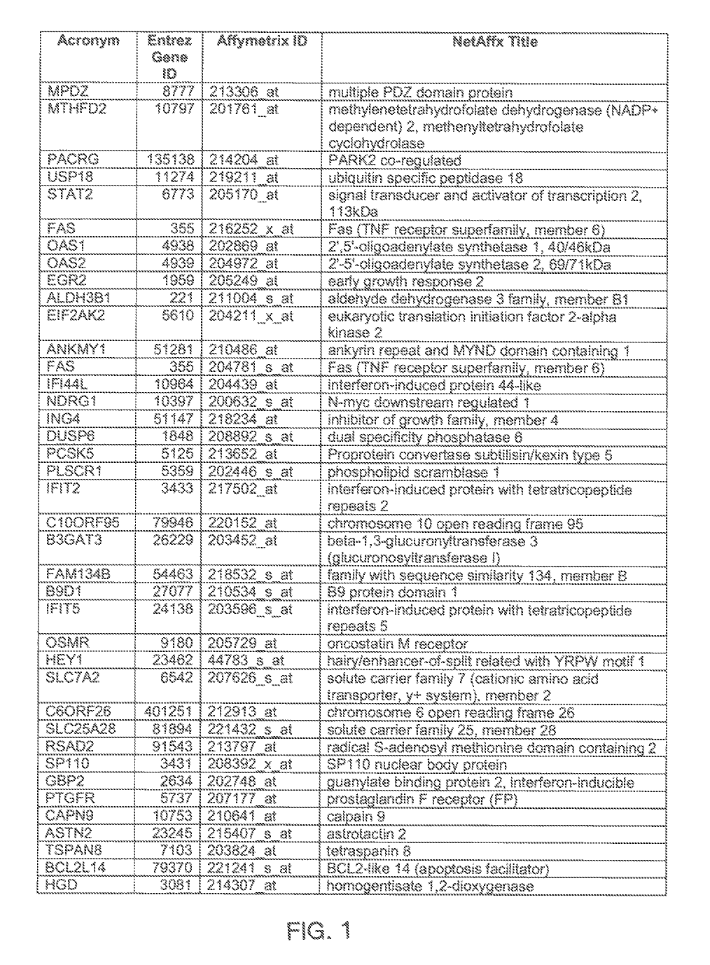

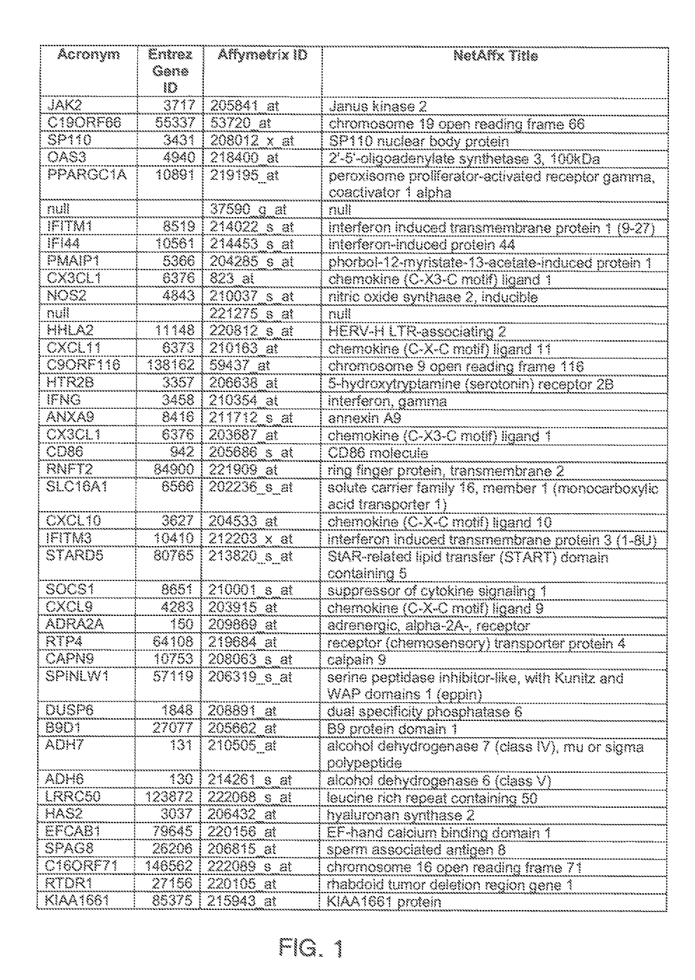

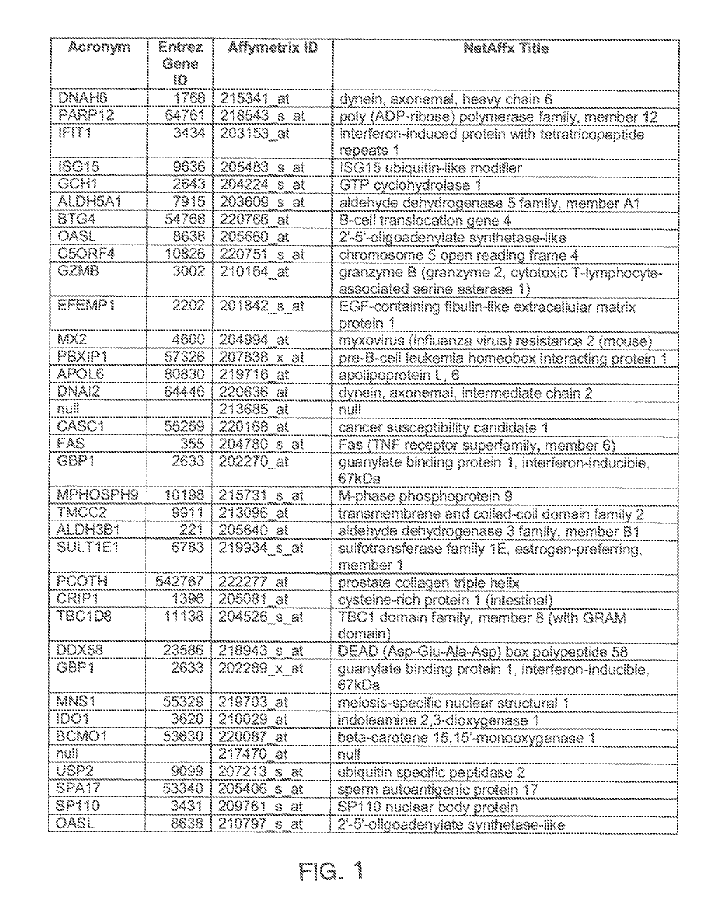

The invention is predicated, at least in part, on the identification of genes with enhanced (up-regulated) or diminished (down-regulated) expression representative of rhinovirus infection. As described in greater detail in the Examples, a number of gene transcripts were identified that are present in increased or decreased levels in individuals infected with rhinovirus. A list of the gene transcripts is provided in FIG. 1. A subset of the gene transcripts in FIG. 1 is set forth in Table 1 below, which provides the acronym and description of the protein corresponding to the identified gene transcript, the Affymetrix probe identifier from AFFYMETRIX_{—}3PRIME_IVT_ID (Affymetrix GeneChip® Human Genome U133 (HG-U133) Plus 2.0 Array (by Affymetrix, Inc. Santa Clara, Calif. 95051 USA) that binds the gene transcript, and the Entrez Gene identifier (NCBI, U.S. National Library of Medicine, Bethesda, Md.).

TABLE 1

| | | Entrez | |
|---|---|---|---|
| Name | Affymetrix ID | Gene ID | Description |
| ACCN2 | 205156_s_at | 41 | amiloride-sensitive cation channel 2, |
| | | | neuronal |
| ADCY2 | 213217_at | 108 | adenylate cyclase 2 (brain) |
| ADH6 | 214261_s_at | 130 | alcohol dehydrogenase 6 (class V) |
| ALDH3B1 | 211004_s_at, | 221 | aldehyde dehydrogenase 3 family, member B1 |
| | 205640_at | | |
| ALDH5A1 | 203609_s_at | 7915 | aldehyde dehydrogenase 5 family, member A1 |
| B3GAT3 | 203452_at | 26229 | beta-1,3-glucuronyltransferase 3 |
| | | | (glucuronosyltransferase I) |
| BATF3 | 220358_at | 55509 | basic leucine zipper transcription factor, |
| | | | ATF-like 3 |
| BCL2L14 | 221241_s_at | 79370 | BCL2-like 14 (apoptosis facilitator) |
| BTBD3 | 202946_s_at | 22903 | BTB (POZ) domain containing 3 |
| BTG4 | 220766_at | 54766 | B-cell translocation gene 4 |
| C10ORF95 | 220152_at | 79946 | chromosome 10 open reading frame 95 |
| C11ORF16 | 220344_at | 56673 | chromosome 11 open reading frame 16 |
| C19ORF66 | 53720_at | 55337 | chromosome 19 open reading frame 66 |
| C5ORF4 | 220751_s_at | 10826 | chromosome 5 open reading frame 4 |
| C5ORF42 | 219381_at | 65250 | chromosome 5 open reading frame 42 |
| C7ORF63 | 219455_at | 79846 | chromosome 7 open reading frame 63 |
| C9ORF116 | 59437_at, | 138162 | chromosome 9 open reading frame 116 |
| | 221946_at | | |
| CAPN9 | 210641_at, | 10753 | calpain 9 |
| | 208063_s_at | | |
| CASC1 | 220168_at | 55259 | cancer susceptibility candidate 1 |
| CCDC81 | 220389_at | 60494 | coiled-coil domain containing 81 |
| CDC14A | 205288_at | 8556 | CDC14 cell division cycle 14 homolog A |
| | | | (S. cerevisiae) |
| CLEC2B | 209732_at | 9976 | C-type lectin domain family 2, member B |
| CRY2 | 212695_at | 1408 | cryptochrome 2 (photolyase-like) |
| CX3CL1 | 823_at | 6376 | chemokine (C-X3-C motif) ligand 1 |
| CXCL10 | 204533_at | 3627 | chemokine (C-X-C motif) ligand 10 |
| CXCL11 | 210163_at, | 6373 | chemokine (C-X-C motif) ligand 11 |
| | 211122_s_at | | |
| CXCL9 | 203915_at | 4283 | chemokine (C-X-C motif) ligand 9 |
| DDX60 | 218986_s_at | 55601 | DEAD (Asp-Glu-Ala-Asp) box polypeptide 60 |
| DNAH6 | 215341_at | 1768 | dynein, axonemal, heavy chain 6 |
| ETV7 | 221680_s_at | 51513 | ets variant 7 |
| FAM134B | 218532_s_at | 54463 | family with sequence similarity 134, |
| | | | member B |
| FAS | 204781_s_at, | 355 | Fas (TNF receptor superfamily, member 6) |
| | 215719_x_at, | | |
| | 204780_s_at | | |
| GBP1 | 202270_at, | 2633 | guanylate binding protein 1, interferon- |
| | 202269_x_at | | inducible, 67 kDa |
| GCH1 | 204224_s_at | 2643 | GTP cyclohydrolase 1 |
| GOLGA2B | 219876_s_at | 55592 | golgin A2 family, member B |
| GZMB | 210164_at | 3002 | granzyme B (granzyme 2, cytotoxic T- |
| | | | lymphocyte-associated serine esterase 1) |
| HAS2 | 206432_at | 3037 | hyaluronan synthase 2 |
| HERC6 | 219352_at | 55008 | hect domain and RLD 6 |
| HEY1 | 44783_s_at, | 23462 | hairy/enhancer-of-split related with YRPW |
| | 218839_at | | motif 1 |
| HHLA2 | 220812_s_at | 11148 | HERV-H LTR-associating 2 |
| IFI44 | 214453_s_at | 10561 | interferon-induced protein 44 |
| IFI44L | 204439_at | 10964 | interferon-induced protein 44-like |
| IFIH1 | 219209_at | 64135 | interferon induced with helicase C domain 1 |
| IFIT1 | 203153_at | 3434 | interferon-induced protein with |
| | | | tetratricopeptide repeats 1 |
| IFIT2 | 217502_at | 3433 | interferon-induced protein with |
| | | | tetratricopeptide repeats 2 |
| IFIT3 | 204747_at | 3437 | interferon-induced protein with |
| | | | tetratricopeptide repeats 3 |
| IFIT5 | 203596_s_at | 24138 | interferon-induced protein with |
| | | | tetratricopeptide repeats 5 |
| IFITM1 | 201601_x_at | 8519 | interferon induced transmembrane protein 1 |
| | | | (9-27) |
| IQCH | 220361_at | 64799 | IQ motif containing H |
| ISG15 | 205483_s_at | 9636 | ISG15 ubiquitin-like modifier |
| LRP2BP | 207797_s_at | 55805 | LRP2 binding protein |
| LRRC23 | 206076_at | 10233 | leucine rich repeat containing 23 |
| LRRC36 | 220003_at | 55282 | leucine rich repeat containing 36 |
| LRRC50 | 222068_s_at | 123872 | leucine rich repeat containing 50 |
| MX1 | 202086_at | 4599 | myxovirus (influenza virus) resistance 1, |
| | | | interferon-inducible protein p78 (mouse) |
| MX2 | 204994_at | 4600 | myxovirus (influenza virus) resistance 2 |
| | | | (mouse) |
| NDRG1 | 200632_s_at | 10397 | N-myc downstream regulated 1 |
| OAS2 | 204972_at, | 4939 | 2′-5′-oligoadenylate synthetase 2, |
| | 206553_at | | 69/71 kDa |
| OSMR | 205729_at | 9180 | oncostatin M receptor |
| PACRG | 214204_at | 135138 | PARK2 co-regulated |
| PCOTH | 222277_at | 542767 | prostate collagen triple helix |
| PCSK5 | 213652_at | 5125 | Proprotein convertase subtilisin/kexin type 5 |
| PLSCR1 | 202446_s_at | 5359 | phospholipid scramblase 1 |
| PMAIP1 | 204285_s_at, | 5366 | phorbol-12-myristate-13-acetate-induced |
| | 204286_s_at | | protein 1 |
| PML | 209640_at | 5371 | promyelocytic leukemia |
| PSD3 | 218613_at | 23362 | pleckstrin and Sec7 domain containing 3 |
| PTGFR | 207177_at | 5737 | prostaglandin F receptor (FP) |
| RAB3B | 205924_at, | 5865 | RAB3B, member RAS oncogene family |
| | 205925_s_at | | |
| RNFT2 | 221908_at, | 84900 | ring finger protein, transmembrane 2 |
| | 221909_at | | |
| SLC16A1 | 202236_s_at | 6566 | solute carrier family 16, member 1 |
| | | | (monocarboxylic acid transporter 1) |
| SLC25A28 | 221432_s_at | 81894 | solute carrier family 25, member 28 |
| SOCS1 | 209999_x_at, | 8651 | suppressor of cytokine signaling 1 |
| | 210001_s_at | | |
| SP110 | 208392_x_at, | 3431 | SP110 nuclear body protein |
| | 209761_s_at, | | |
| | 208012_x_at, | | |
| | 209762_x_at | | |
| SPAG8 | 206815_at | 26206 | sperm associated antigen 8 |
| STARD5 | 213820_s_at | 80765 | StAR-related lipid transfer (START) |
| | | | domain containing 5 |
| SULT1E1 | 219934_s_at | 6783 | sulfotransferase family 1E, estrogen- |
| | | | preferring, member 1 |
| TBC1D8 | 204526_s_at | 11138 | TBC1 domain family, member 8 (with |
| | | | GRAM domain) |
| TRIM22 | 213293_s_at | 10346 | tripartite motif-containing 22 |
| TRIM3 | 213884_s_at | 10612 | tripartite motif-containing 3 |
| TSPAN8 | 203824_at | 7103 | tetraspanin 8 |
| USP2 | 207213_s_at | 9099 | ubiquitin specific peptidase 2 |
| XAF1 | 206133_at | 54739 | XIAP associated factor 1 |

The invention provides, e.g., a method for evaluating the activity of an agent for treating rhinovirus infection or a symptom thereof, a method of detecting or monitoring rhinovirus infection in a human patient, and a method of treating rhinovirus infection or a symptom thereof comprising measuring expression of a panel of genes (e.g., any and all combinations of two or more of the genes identified herein) to generate a gene expression profile (i.e., a collection of measurements of gene expression). While a gene expression profile may represent all genes significantly regulated in response to rhinovirus infection, typically it represents a subset of such genes. Thus, in various aspects, the panel of genes (also referred to herein as a “gene panel”) comprises at least two, at least four, at least five, at least 10, at least 20, at least 25, at least 30, or at least 50 genes (e.g., 75 or more genes). Alternatively or in addition, the panel of genes includes no more than 10,000, no more than 7,500, no more than 5,000, no more than 1,000, no more than 500, no more than 300, no more than 250, no more than 200, no more than 150, no more than 100 genes, no more than 82 genes, no more than 50 genes, no more than 26 genes, or no more than 20 genes. Preferred ranges of the number of genes, the expression of which is measured, include 2-5, 2-10, 2-50, 2-75, 2-100, 5-10, 5-20, 5-50, 5-75, 5-100, 10-20, 10-50, 10-75, 10-100, 20-50, 20-75, 20-100, 26-500, 26-250, 26-200, 26-100, 26-82, 50-75, 50-100, 100-125, 125-150, 150-175, and 175-200, although a gene panel can comprise from 200-250 genes, 250-300 genes, 300-350 genes, 350-400 genes, 400-450 genes, 450-500 genes, 500-550 genes, 550-600 genes, 600-650 genes, 650-700 genes, 700-750 genes, 750-800 genes, 2-10,000 genes, 100-10,000 genes, 1,000-10,000 genes, 1,000-5,000 genes, or 5,000-10,000 genes. In some aspects, the gene panel is limited to the genes set forth in Table 1 or FIG. 1, although expression of additional genes may be measured as part of the gene panel in other embodiments of the invention.

Various aspects of the invention comprise measuring expression of a panel of genes from at least one biological sample to produce a gene expression profile, and comparing the gene expression profile to a reference gene expression profile. In multiple embodiments, the gene panel comprises CRY2, B3GAT3, C10ORF95, and/or BATF3 (e.g., CRY2, B3GAT3, C10ORF95, and BATF3), optionally in combination with one or more other genes set forth in FIG. 1, such as one or more of the genes referenced in Table 1. Optionally, the gene panel comprises (i) one or more of CRY2, B3GAT3, C10ORF95, and/or BATF3 and (ii) one or more (e.g., all) of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. In one aspect, the method comprises measuring expression of five or more, 10 or more, or all of the genes (i.e., any combination of five (or 10) or more of CRY2, B3GAT3, C10ORF95, BATF3, RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, so long as the panel comprises at least one of CRY2, B3GAT3, C10ORF95, and/or BATF3). Optionally, the panel further comprises one or more (e.g., 10 or more) additional genes selected from the group consisting of ACCN2, ADCY2, ADH6, ALDH5A1, BCL2L14, BTBD3, C11ORF16, C19ORF66, C5ORF4, C5ORF42, C7ORF63, C9ORF116, CASC1, CCDC81, CDCl₄A, CX3CL1, DDX60, DNAH6, ETV7, GCH1, GOLGA2B, GZMB, HERC6, HHLA2, IF144, IF144L, IFIH1, IFIT2, IFIT5, IFITM1, IQCH, ISG15, LRP2BP, LRRC23, LRRC50, MX2, NDRG1, OAS2, OSMR, PACRG, PCOTH, PCSK5, PMAIP1, PML, PTGFR, SLC16A1, SLC25A28, SOCS1, SPAG8, STARD5, TBC1D8, TRIM22, TRIM3, TSPAN8, USP2, and XAF1. Any combination of genes is suitable for a gene panel. Put another way, the inventive method in various aspects comprises measuring expression of (i) one or more genes selected from the group consisting of CRY2, B3GAT3, C10ORF95, and BATF3 and (ii) one or more (e.g., any combination of five or ten or more) genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, and, optionally, (iii) one or more (e.g., any combination of five or ten or more) genes selected from the group consisting of ACCN2, ADCY2, ADH6, ALDH5A1, BCL2L14, BTBD3, C11ORF16, C19ORF66, C5ORF4, C5ORF42, C7ORF63, C9ORF116, CASC1, CCDC81, CDCl₄A, CX3CL1, DDX60, DNAH6, ETV7, GCH1, GOLGA2B, GZMB, HERC6, HHLA2, IF144, IF144L, IFIH1, IFIT2, IFIT5, IFITM1, IQCH, ISG15, LRP2BP, LRRC23, LRRC50, MX2, NDRG1, OAS2, OSMR, PACRG, PCOTH, PCSK5, PMAIP1, PML, PTGFR, SLC16A1, SLC25A28, SOCS1, SPAG8, STARD5, TBC1D8, TRIM22, TRIM3, TSPAN8, USP2, and XAF1. In any embodiment, the invention comprises measuring expression of all of the genes referenced in Table 1. A gene panel can also comprise additional genes associated with rhinovirus infection in combination with a subset or all of the genes listed in FIG. 1 or Table 1.

### Measuring Gene Expression

In various aspects, the invention comprises measuring gene expression in at least one biological sample of an animal, such as a human, to produce a gene expression profile. Typical biological samples include, but are not limited to, sputum, nasal wash (lavage), nasal swab, nasal aspirate, oral swab, saliva, blood, tissue biopsy, peritoneal fluid, and pleural fluid. The biological sample is obtained from a subject using any clinically-accepted method. For example, nasal lavage samples are collected by instillation of, e.g., saline solution into each nostril. The wash is immediately expelled into a receptacle and prepared for analysis. Nasal scraping samples are collected, for example, from the anterior portion of the inferior turbinate. If desired, nucleic acid and/or protein is at least partially isolated from the biological sample prior to measurement. Many isolation and purification methods are known in the art, such as those described in detail in Chapter 3 of Tijssen, (1993) Laboratory Techniques in Biochemistry and Molecular Biology: Hybridization with Nucleic Acid Probes, Elsevier Press.

Gene expression is detected and/or measured in a variety of ways. Exemplary biomolecules representative of gene expression (i.e., “biomarkers”) include protein, nucleic acid (e.g., mRNA or cDNA), protein fragments or metabolites, and/or products of enzymatic activity encoded by the protein encoded by a gene transcript, and detection and/or measurement of any of the biomarkers described herein is suitable in the context of the invention. In one embodiment, the method comprises measuring mRNA encoded by one or more of the genes. If desired, the method comprises reverse transcribing mRNA encoded by one or more of the genes and measuring the corresponding cDNA. Any quantitative nucleic acid assay may be used. For example, many quantitative hybridization, Northern blot, and polymerase chain reaction procedures exist for quantitatively measuring the amount of an mRNA transcript or cDNA in a biological sample. See, e.g., Current Protocols in Molecular Biology, Ausubel et al., eds., John Wiley & Sons (2007), including all supplements. Optionally, the mRNA or cDNA is amplified by polymerase chain reaction (PCR) prior to hybridization. The mRNA or cDNA sample is then examined by, e.g., hybridization with oligonucleotides specific for mRNAs or cDNAs encoded by one or more of the genes of the panel, optionally immobilized on a substrate (e.g., an array or microarray). Selection of one or more suitable probes specific for an mRNA or cDNA, and selection of hybridization or PCR conditions, are within the ordinary skill of scientists who work with nucleic acids. Binding of the biomarker nucleic acid to oligonucleotide probes specific for the biomarker(s) allows identification and quantification of the biomarker.

The invention also contemplates measuring protein encoded by one or more of the genes to generate a gene expression profile. Any technique for quantifying a protein may be used for quantifying proteins in the context of the invention. For example, quantitative mass spectrometry is suitable for measuring protein in a sample, including measuring small amounts of protein in a small sample. Numerous antibody-based methods exist for quantifying proteins in samples, including Western blot techniques and ELISA assays. For biomarkers having biological activity (e.g., enzymatic activity), measurement of the activity of one or more biomarkers may be used as a surrogate for measuring gene expression. In a typical enzymatic activity assay, for example, a biological sample or fraction thereof is contacted with a substrate for the enzyme under conditions suitable for enzymatic activity, and product of the enzymatic reaction is measured over time.

In some variations, a protein is identified and/or quantified with an immunoassay, using one or more antibodies that preferentially bind, and preferably bind with high specificity, to a protein of interest. Exemplary immunoassays include immunofluorescent immunoassays, immunoprecipitations, radioimmunoasays, ELISA, and Western blotting. The epitope(s) used for recognizing and quantifying a protein may be a linear peptide epitope, a conformational epitope, an epitope that includes one or more side-chain modifications (e.g., glycosylation), and so on. See generally E. Maggio, Enzyme-Immunoassay, (1980) (CRC Press, Inc., Boca Raton, Fla.). See also U.S. Pat. Nos. 4,727,022; 4,659,678; 4,376,110; 4,275,149; 4,233,402, and 4,230,767.

Measurements of gene expression produce a gene expression profile, which contains data representative of the expression levels of two or more of the genes identified herein. In some embodiments, a gene expression profile may comprise a first list representative of a plurality of genes up-regulated during rhinovirus infection (or in response to exposure to an agent, as described further below) and a second list representative of a plurality of genes down-regulated during rhinovirus infection (or in response to exposure to an agent). The gene expression profile is, in various embodiments of the invention, compared to a reference gene expression profile. In one aspect, the reference gene expression profile contains expression measurements of the genes from a similar biological sample taken from, e.g., an animal (such as a human) that is not infected with rhinovirus or from in vitro or ex vivo cells that were not infected with rhinovirus (referred to herein as a “reference gene expression profile of non-infection”). For example, the reference gene expression profile is generated from a biological sample from the animal (e.g., human subject) taken earlier in time, prior to expected exposure to rhinovirus; a control subject or population whose gene expression measurements are known; or an index value or baseline value. In another aspect, the reference gene expression profile contains expression measurements of the genes from a similar biological sample taken from, e.g., an animal (such as a human) or population of animals (e.g., humans) exposed to rhinovirus (referred to herein as a “reference gene expression profile of infection”). A reference gene expression profile can also comprise a value derived from prediction algorithms or computed indices from population studies. In various embodiments, the reference gene expression profile is matched for race, gender, age, geographic location, and/or ethnic origin with respect to the subject.

### Methods of Using a Gene Expression Profile

In one aspect, the invention includes a method of detecting or monitoring rhinovirus infection (e.g., a human rhinovirus infection, such as HRV16 infection) in a human patient. The method comprises measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11 from at least one biological sample from a human patient to produce a gene expression profile. The method further comprises comparing the gene expression profile from the patient to a reference gene expression profile. A gene expression profile that (1) differs from a reference gene expression profile of non-infection or (2) does not significantly differ from a reference gene expression profile of infection is indicative of rhinovirus infection. For example, a gene expression profile having (1) a decrease in RNFT2, CRY2, C10ORF95, BTG4, PSD3, CAPN9, SULT1E1, HEY1, LRRC36, RAB3B, ALDH3B1, FAM134B, and/or B3GAT3, and/or (2) an increase in FAS, PLSCR1, CLEC2B, BATF3, HAS2, MX1, SP110, GBP1, IFIT3, IFIT1, CXCL9, CXCL10, and/or CXCL11 compared to a reference gene expression profile of non-infection is indicative of rhinovirus infection. In any of the embodiments described herein, a decrease in gene expression of RNFT2, CRY2, C10ORF95, BTG4, PSD3, CAPN9, SULT1E1, HEY1, LRRC36, RAB3B, ALDH3B1, FAM134B, and/or B3GAT3 that is at least about a 0.25-fold change (e.g., at least about a 0.5-fold or at least about a 0.75-fold) change in expression compared to expression levels observed in a reference gene expression profile of non-infection is indicative (predictive) of infection. In any of the embodiments described herein, an increase in FAS, PLSCR1, CLEC2B, BATF3, HAS2, MX1, SP110, GBP1, IFIT3, IFIT1, CXCL9, CXCL10, and/or CXCL11 that is at least about a 1.5-fold increase (e.g., at least about a 2.0-fold increase or at least about a 3.0-fold increase) in expression compared to expression levels observed in a reference gene expression profile of non-infection is indicative (predictive) of infection. The gene expression profile alternatively (or additionally) may be compared to a reference gene expression profile of infection. In this regard, a subject gene expression profile demonstrating (1) increases in RNFT2, CRY2, C10ORF95, BTG4, PSD3, CAPN9, SULT1E1, HEY1, LRRC36, RAB3B, ALDH3B1, FAM134B, and/or B3GAT3, and/or (2) decreases in FAS, PLSCR1, CLEC2B, BATF3, HAS2, MX1, SP110, GBP1, IFIT3, IFIT1, CXCL9, CXCL10, and/or CXCL11 compared to an reference gene expression profile of infection is indicative of absence or amelioration (i.e., improvement) of infection. A subject gene expression profile having (1) undetectable or insignificant increases in RNFT2, CRY2, C10ORF95, BTG4, PSD3, CAPN9, SULT1E1, HEY1, LRRC36, RAB3B, ALDH3B1, FAM134B, and/or B3GAT3, and/or (2) undetectable or insignificant decreases in FAS, PLSCR1, CLEC2B, BATF3, HAS2, MX1, SP110, GBP1, IFIT3, IFIT1, CXCL9, CXCL10, and/or CXCL11 compared to an reference gene expression profile of infection is indicative of infection. Any mode of statistical analysis is appropriate for determining statistical significance of an increase or decrease in expression levels, e.g., the T-test. The method further optionally comprises determining a probability of achieving a therapeutic response to an agent from an output of a model, wherein inputs to the model comprise expression measurements.

Optionally, the method is employed to distinguish rhinovirus infection from other viral infections. An exemplary gene panel of the invention, RNFT2, CRY2, C10ORF95, BTG4, PSD3, CAPN9, SULT1E1, HEY1, LRRC36, RAB3B, ALDH3B1, FAM134B, B3GAT3, FAS, PLSCR1, CLEC2B, BATF3, HAS2, MX1, SP110, GBP1, IFIT3, IFIT1, CXCL9, CXCL10, and CXCL11, comprises no overlapping genes with the gene signature identified for influenza A. See, e.g., Josset L, PLoS One; 5(10): e13169 (2010). Thus, if desired, the method comprises distinguishing rhinovirus infection from other viral infections, including other respiratory viral infections, such as influenza (e.g., influenza A).

The invention also includes a method for evaluating the activity of an agent for treating rhinovirus infection or a symptom thereof. The method comprises (a) administering an agent to an animal infected with rhinovirus, and (b) measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11 from at least one biological sample from the animal to produce a gene expression profile. A gene expression profile that differs from a reference gene expression profile indicates that the agent treats rhinovirus infection or a symptom thereof. In various embodiments, the reference gene expression profile is a reference gene expression profile of infection, and an increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or a decrease in expression of FAS, PLSCR1, CLEC2B, BATF3, HAS2, MX1, SP110, GBP1, IFIT3, IFIT1, CXCL9, CXCL10, and/or CXCL11 in the gene expression profile compared to the reference gene expression profile of infection indicates that the agent treats rhinovirus infection or a symptom thereof. Any increase in RNFT2, CRY2, C10ORF95, BTG4, PSD3, CAPN9, SULT1E1, HEY1, LRRC36, RAB3B, ALDH3B1, FAM134B, and/or B3GAT3 expression, and/or (2) decrease in FAS, PLSCR1, CLEC2B, BATF3, HAS2, MX1, SP110, GBP1, IFIT3, IFIT1, CXCL9, CXCL10, and/or CXCL11 expression is beneficial to the subject. If compared to a reference gene expression profile of non-infection, an undetectable or insignificant decrease in expression of RNFT2, CRY2, C10ORF95, BTG4, PSD3, CAPN9, SULT1E1, HEY1, LRRC36, RAB3B, ALDH3B1, FAM134B, and/or B3GAT3, and/or (2) an undetectable or insignificant increase in expression of FAS, PLSCR1, CLEC2B, BATF3, HAS2, MX1, SP110, GBP1, IFIT3, IFIT1, CXCL9, CXCL10, and/or CXCL11 compared to the uninfected reference gene expression profile indicates that the agent treats rhinovirus infection or a symptom thereof. Animal models of rhinovirus infection are described in the literature. See, e.g., Newcomb and Peebles, Proc Am Thorac Soc, 6(3): 266 (2009); and Bartlett et al., Nature Medicine, 14, 199-204 (2008). The method can be used to screen any agent for activity against rhinovirus (e.g., antiviral activity) or the ability to improve one or more symptoms associated with rhinovirus infection. Exemplary agents include, but are not limited to, natural products, such as plant or mammal extracts; synthetic chemicals; small molecules; peptides; proteins (such as antibodies or fragments thereof); peptidomimetics; and polynucleotides (DNA or RNA). Optionally, the method further comprises scoring the activity of the agent from the output of a model, wherein inputs to the model comprise expression measurements of the genes.

The invention also includes a method of treating rhinovirus infection or a symptom thereof. The method comprises (a) administering to a human patient an agent according to a therapeutic regimen for treating rhinovirus infection or a symptom thereof; (b) measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11 from at least one biological sample from the patient to produce a gene expression profile; and (c) comparing the gene expression profile from the patient to a reference gene expression profile. The method further comprises (d) generating a new therapeutic regimen for the patient to treat rhinovirus infection or a symptom thereof if the gene expression profile (i) differs from a reference gene expression profile of non-infection or (ii) does not significantly differ from a reference gene expression profile of infection. In various aspects, the reference gene expression profile is indicative of non-infection, and a new therapeutic regimen is generated if the subject gene expression profile comprises a decrease in expression RNFT2, CRY2, C10ORF95, BTG4, PSD3, CAPN9, SULT1E1, HEY1, LRRC36, RAB3B, ALDH3B1, FAM134B, and/or B3GAT3, and/or an increase in FAS, PLSCR1, CLEC2B, BATF3, HAS2, MX1, SP110, GBP1, IFIT3, IFIT1, CXCL9, CXCL10, and/or CXCL11, compared to the uninfected reference gene expression profile. Alternatively, the method comprises (d) generating a new therapeutic regimen for the patient to treat rhinovirus infection or a symptom thereof if the gene expression profile comprises an undetectable or insignificant increase in expression RNFT2, CRY2, C10ORF95, BTG4, PSD3, CAPN9, SULT1E1, HEY1, LRRC36, RAB3B, ALDH3B1, FAM134B, and/or B3GAT3, and/or an undetectable or insignificant decrease in FAS, PLSCR1, CLEC2B, BATF3, HAS2, MX1, SP110, GBP1, IFIT3, IFIT1, CXCL9, CXCL10, and/or CXCL11, compared to an reference gene expression profile of infection.

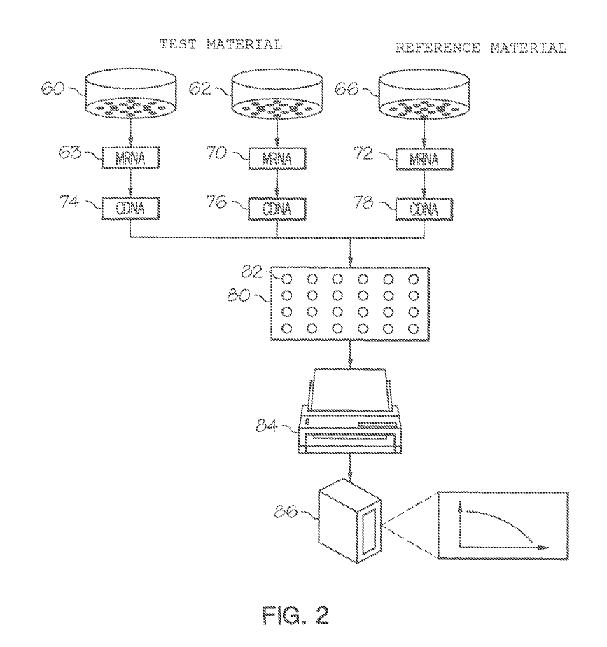

An exemplary method of measuring biomarkers to produce a gene expression profile and comparing the gene expression profile to a reference gene expression profile is described below with reference to FIG. 2. mRNA is extracted from a biological sample and transcribed to cDNA, which is marked with different fluorescent dyes (e.g., red and green) if a two color microarray analysis is performed. Alternatively, the samples are prepped for a one color microarray analysis, and further a plurality of replicates is processed if desired. Optionally, the procedure also is carried out on a reference (control) sample. The cDNA samples are co-hybridized to the microarray 80 comprising a plurality (e.g., tens, hundreds, or thousands) of probes 82. In one aspect, each probe on the microarray has a unique probe set identifier. The microarray is scanned by a scanner 84, which excites the dyes and measures the amount of fluorescence. A computing device 86 analyzes the raw images to determine the amount of cDNA, which is representative of the expression levels of a gene. The scanner 84 may incorporate the functionality of the computing device 86. Gene expression data collected by the system include: i) up-regulation of gene expression (e.g., greater binding of the test material (e.g., cDNA 74, 76) to probes compared to reference material (e.g., cDNA 78)), ii) down-regulation of gene expression (e.g., reduced binding of the test material (e.g., cDNA 74, 76) to probes than the test material (e.g., cDNA 78)), iii) non-fluctuating gene expression (e.g., similar binding of the test material (e.g., cDNA 74, 76) to the probes compared to the reference material (e.g., cDNA 78)), and iv) no detectable signal or noise.

Microarrays and microarray analysis techniques are well known in the art, and it is contemplated that other microarray techniques may be used with the methods, devices, and systems of the invention. For example, any suitable commercial or non-commercial microarray technology and associated techniques may used, such as, but not limited to Affymetrix GeneChip™ technology and Illumina BeadChip™ technology. One of skill in the art will appreciate that the invention is not limited to the methodology described above, and that other methods and techniques are also contemplated to be within its scope of the invention.

### Kits

The invention further provides a kit containing reagents packaged together and useful for measuring expression of a panel of genes as described herein. In various aspects, the gene panel consists of 2 to 10,000 genes (e.g., 4 to 10,000 genes, 4 to 5,000 genes, 10 to 5,000 genes, 20 to 1,000 genes, 20 to 500 genes, 26 to 500 genes, 26 to 300 genes, 26 to 200 genes, 26 to 100 genes, 26 to 82 genes, or 26 to 50 genes), and comprises (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. For example, the kit may contain, in separate containers but packaged together, one or more oligonucleotide probes that hybridize to polynucleotides of interest (e.g., mRNA encoded by one or more genes or cDNA reverse transcribed from the mRNA), and reagents for performing a hybridization-based quantitative assay or a PCR-based quantitative assay. The kit optionally further includes molecular standards and/or positive and/or negative control formulations for the polynucleotides to be screened. In various embodiments, the kit comprises primers for amplification of mRNA encoded by one or more of the genes or cDNA reverse transcribed from the mRNA.

One of skill in the art has the requisite knowledge and skill to design primers for amplifying a nucleic acid of interest as well as oligonucleotide probes for sequence-specific hybridization to a desired nucleic acid, and probes specific to the polynucleotide biomarkers described herein are commercially available. By “sequence-specific hybridization” is meant that the probe(s) preferentially bind to a nucleic acid sequence encoding a target nucleic acid. In some embodiments, specific hybridization is achieved using “stringent conditions,” which are conditions for hybridization and washing under which nucleotide sequences at least 60% complementary to each other typically remain hybridized. It is appreciated in the art that stringent conditions can differ depending on sequence content, probe length, and the like. A non-limiting example of stringent hybridization conditions are hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2X SSC, 0.01% BSA at 50° C.

Specific hybridization, if present, is detected using any suitable detection method, and the kit optionally contains one or more reagents for detection. For example, the probe can comprise a fluorescent moiety at its 3′ terminus, a quencher at its 5′ terminus, and an enhancer oligonucleotide to facilitate detection, as described by Kutyavin et al., Nucleic Acid Res. 34:e128 (2006). In this detection method, an enzyme cleaves the fluorescent moiety from a fully complementary detection probe, but does not cleave the fluorescent moiety if the probe contains a mismatch. The presence of a particular target sequence is signaled by the fluorescence of the released fluorescent moiety. Alternatively or in addition, a solid support comprising one or more probes (e.g., an array) attached thereto is employed. Oligonucleotide arrays typically comprise a plurality of different oligonucleotide probes coupled to a surface of a substrate (e.g., plastic, complex carbohydrate, or acrylic resin) in different known locations. Methods of producing arrays are known to the ordinary skilled practitioner (see, e.g., Bier et al., Adv. Biochem. Eng. Biotechnol., 109:433-53 (2008); Hoheisel, Nat. Rev. Genet., 7:200-10 (2006); Fan et al., Methods Enzymol., 410:57-73 (2006); Raqoussis & Elvidge, Expert Rev. Mol. Diagn., 6:145-52 (2006); Mockler et al., Genomics, 85:1-15 (2005), and references cited therein, the entire teachings of each of which are incorporated by reference herein). The location of probes specific to a particular gene transcript is cataloged, hybridization to the immobilized probe is detected, and the polynucleotide is identified by the location of the hybridization on the array.

Alternatively or in addition, the kit comprises one or more antibodies or antigen-binding fragments thereof, such as a monoclonal antibody or antigen-binding fragment thereof, specific for one or more biomarkers of interest (e.g., protein encoded by one or more of the genes of the gene panel). In some variations, the antibody is pre-bound to a solid matrix such as a plate or bead. In other variations, the kit may include reagents to attach an antibody to a solid matrix, such as such as plastic, nylon, or paper. Optionally, the kit further includes one or more detectable labels (e.g., fluorescent or luminescent tags, metals, dyes, or radionuclides) and/or secondary antibodies for quantifying binding between the primary antibody and the biomarker. Labels for indirect detection of antibody-target binding include enzymes or enzyme substrates such as, e.g., alkaline phosphatase and horseradish peroxidase.

The kit optionally further includes instructions for determining the presence or severity of rhinovirus infection. The kit also optionally comprises buffers, enzymes (e.g., horseradish peroxidase, alkaline phosphatase, etc.), enzyme substrates, wash reagents, and the like, packaged together or in separate containers. Further, it will be appreciated that analysis can be carried out in a variety of physical formats, and the format of the kit will depend on the detection method(s) and setting of use. For example, assay plates or large arrays are suitable for processing large numbers of biological samples. Alternatively, single sample formats are suitable for point of care settings or testing a small number of biological samples. In various embodiments, the kit includes one or more tools for collecting a biological sample, such as a cytology collection curette, nasal wash receptacle, or oral swab.

### Systems and Computer-Related Aspects of the Invention

The invention includes a diagnostic test system comprising a data collection tool adapted to collect data representative of expression measurements of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11; an analysis tool comprising a statistical analysis engine adapted to generate a representation of a correlation between the presence or severity of rhinovirus infection and expression measurements of the genes, wherein the representation of the correlation is adapted to be executed to generate a result; and an index computation tool adapted to analyze the result to determine the presence or severity of rhinovirus infection and represent the result as a numerical probability or a grade or score. The system optionally further comprises a reporting tool adapted to generate a report comprising the numerical probability, grade, or score. In various embodiments, the data collection tool is adapted to collect data representative of expression measurements of four or more of the genes, five or more of the genes, 10 or more of the genes, or all of the genes. Additionally, the data collection tool is optionally adapted to collect data representative of expression measurements of one or more additional genes (e.g., 10 or more of additional genes) selected from the group consisting of ACCN2, ADCY2, ADH6, ALDH5A1, BCL2L14, BTBD3, C11ORF16, C19ORF66, C5ORF4, C5ORF42, C7ORF63, C9ORF116, CASC1, CCDC81, CDCl₄A, CX3CL1, DDX60, DNAH6, ETV7, GCH1, GOLGA2B, GZMB, HERC6, HHLA2, IF144, IFI44L, IFIH1, IFIT2, IFIT5, IFITM1, IQCH, ISG15, LRP2BP, LRRC23, LRRC50, MX2, NDRG1, OAS2, OSMR, PACRG, PCOTH, PCSK5, PMAIP1, PML, PTGFR, SLC16A1, SLC25A28, SOCS1, SPAG8, STARD5, TBC1D8, TRIM22, TRIM3, TSPAN8, USP2, and XAF1. In one aspect, the data collection tool is adapted to collect data representative of expression measurements of all of the additional genes.

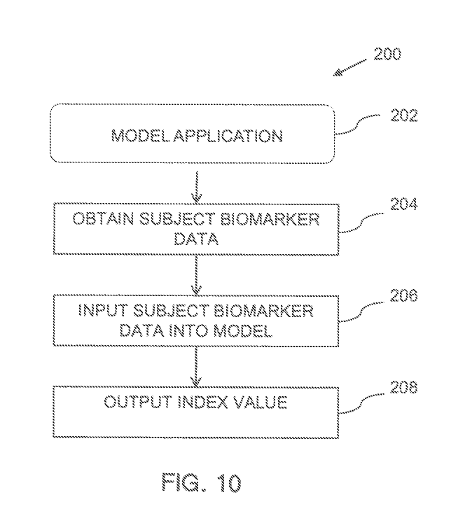

FIG. 10 is a flow diagram of an example method 200 for using a model based on the gene expression signature of rhinovirus described herein to evaluate the presence or severity of rhinovirus infection in a subject (e.g., a person, or group of people). At a block 204, biomarker data from the subject is obtained from a data collection tool, data storage medium, or a data storage system (described further below). The subject biomarker data may be initially derived through a variety of means, including measuring from one or more biological samples; self-reports; physical examination; laboratory testing; existing medical records, charts or databases; and combinations thereof. The subject biomarker data at block 206 may be prepared using transforms, logs, combinations, normalization, etc., as needed according to the model, and input into the model for analysis. At block 208 the analysis tool and index computation tool outputs an index value (e.g., probability of infection, or score/grade representative of probability of infection, etc.).

The invention further includes a computer readable medium having computer executable instructions for determining the presence or severity of rhinovirus infection. The computer readable medium comprises (a) a routine, stored on the computer readable medium and adapted to be executed by a processor, to store expression measurement data representing expression measurements of a gene panel. Optionally, the routine to store expression measurement data also stores clinical measurement data including data representing measurements of at least one clinical parameter selected from the group consisting of sneezing, malaise, chilliness, nasal discharge, swelling of mucosal membranes, wheezing, sore throat, hoarseness, coughing, watery eyes, headache, body ache, ear ache or inflammation, sinusitis, and fever. The computer readable medium further comprises (b) a routine, stored on the computer readable medium and adapted to be executed by a processor, to analyze the expression measurement data to evaluate the presence or severity of rhinovirus infection. The gene panel comprises (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. In various embodiments, the gene panel comprises five or more of the genes, 10 or more of the genes, or all of the genes. Additionally, the gene panel optionally includes one or more additional genes (e.g., 10 or more of additional genes) selected from the group consisting of ACCN2, ADCY2, ADH6, ALDH5A1, BCL2L14, BTBD3, C11ORF16, C19ORF66, C5ORF4, C5ORF42, C7ORF63, C9ORF116, CASC1, CCDC81, CDCl₄A, CX3CL1, DDX60, DNAH6, ETV7, GCH1, GOLGA2B, GZMB, HERC6, HHLA2, IF144, IF144L, IFIH1, IFIT2, IFIT5, IFITM1, IQCH, ISG15, LRP2BP, LRRC23, LRRC50, MX2, NDRG1, OAS2, OSMR, PACRG, PCOTH, PCSK5, PMAIP1, PML, PTGFR, SLC16A1, SLC25A28, SOCS1, SPAG8, STARD5, TBC1D8, TRIM22, TRIM3, TSPAN8, USP2, and XAF1. In one aspect, the gene panel comprises all of the additional genes.

A machine-readable storage medium can comprise a data storage material encoded with machine readable data or data arrays which, when using a machine programmed with instructions for using said data, is capable of use for a variety of purposes, such as determining the presence or severity of rhinovirus infection. Measurements of gene expression and/or, for example, the resulting evaluation of efficacy of a candidate therapeutic agent from those gene expression measurements can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein.

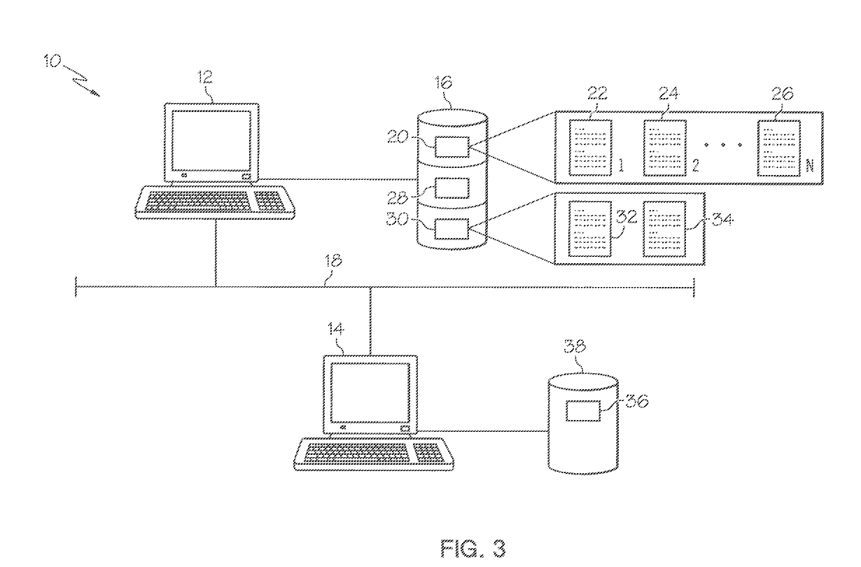

FIG. 3 illustrates an example of a suitable computing system environment 10, 12 on which a system for the steps of the claimed method and apparatus may be implemented. The computing system environment 10 is only one example of a suitable computing environment and is not intended to suggest any limitation as to the scope of use or functionality of the method of apparatus of the claims. Neither should the computing environment 10 be interpreted as having any dependency or requirement relating to any one or combination of components illustrated in the exemplary operating environment 10.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like, including those systems, environments, configurations and means described elsewhere within this disclosure.

While the system is described with respect to gene expression profiles generated from microarray analysis, the description is to be construed as exemplary only. System 10 comprises one or more of computing devices 12, 14, a computer readable medium 16 associated with the computing device 12, and communication network 18. Computer readable media can be any available media that can be accessed by computer and includes both volatile and nonvolatile media, removable and non-removable media. By way of example, and not limitation, computer readable media may comprise computer storage media and communication media. Computer readable storage media include, e.g., application-specific integrated circuit (ASIC), a compact disc (CD), a digital versatile disk (DVD), a random access memory (RAM), a synchronous RAM (SRAM), a dynamic RAM (DRAM), a synchronous DRAM (SDRAM), double data rate SDRAM (DDR SDRAM), a direct RAM bus RAM (DRRAM), a read only memory (ROM), a programmable read only memory (PROM), an electronically erasable programmable read only memory (EEPROM), a disk, a memory stick, or other physical storage elements that physically embody electronic data and excludes any propagated media such as radio waves or modulated carrier signals. Examples of volatile memory include, but are not limited to, random access memory (RAM), synchronous RAM (SRAM), dynamic RAM (DRAM), synchronous DRAM (SDRAM), double data rate SDRAM (DDR SDRAM), and direct RAM bus RAM (DRRAM). Examples of non-volatile memory include, but are not limited to, read only memory (ROM), programmable read only memory (PROM), erasable programmable read only memory (EPROM), and electrically erasable programmable read only memory (EEPROM). A memory can store processes and/or data. Still other computer readable media include any suitable disk media, including but not limited to, magnetic disks, floppy disks, tape drives, Zip drives, flash memory, memory sticks, compact disk ROM (CD-ROM), CD recordable drive (CD-R drive), CD rewriteable drive (CD-RW drive), and digital versatile ROM drive (DVD ROM), other optical disk storage or magnetic storage devices, or any other medium which can be used to store the desired information and which can accessed by computer. The machine-readable media can also contain information relating to other algorithms and computed indices.

Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The computer readable medium 16, which may be provided as a hard disk drive, comprises a digital file 20, such as a database file, optionally comprising one or more gene expression profiles 22, 24, and 26 stored in a data structure associated with the digital file 20. The gene expression profiles may be stored in relational tables and indexes or in other types of computer readable media. The gene expression profiles 22, 24, and 26 may also be distributed across a plurality of digital files, a single digital file 20 being described herein however for simplicity.

The digital file 20 can be provided in wide variety of formats, including but not limited to a word processing file format (e.g., Microsoft Word), a spreadsheet file format (e.g., Microsoft Excel), and a database file format. Some common examples of suitable file formats include, but are not limited to, those associated with file extensions such as *.xls, *.xld, *.xlk, *.xll, *.xlt, *.xlxs, *.dif, *.db, *.dbf, *.accdb, *.mdb, *.mdf, *.cdb, *.fdb, *.csv, *sql, *.xml, *.doc, *.txt, *.rtf, *.log, *.docx, *.ans, *.pages, *.wps, etc.

Referring to FIG. 3, in various embodiments, a gene expression profile 22 comprises an ordered listing of microarray probe set IDs. Suitable microarrays include, but are not limited to, Affymetrix® GeneChips and Illumina BeadChips®, both of which comprise probe sets and custom probe sets. Gene expression profiles derived from microarray analyses utilizing Affymetrix GeneChips comprise, in one aspect, an ordered listing of gene probe set IDs, which can comprise as many as over 22,000 IDs. The ordered listing may be stored in a data structure of the digital file 20 and the data arranged so that, when the digital file is read by the software application 28, a plurality of character strings are reproduced representing the ordered listing of probe set IDs. Optionally, the gene expression profile(s) include data in addition to, or in place of, the ordered listing of probe set IDs, such as an ordered listing of equivalent gene names and/or gene symbols. Additional data may be stored with a gene expression profile and/or the digital file 20. In this regard, machine-readable media can also contain additional test results, such as, without limitation, measurements of clinical parameters and traditional laboratory test factors described herein and/or known to clinicians. Alternatively or additionally, the machine-readable media can also comprise subject information such as medical history and any relevant family history. If desired, the ordered list comprises a numeric value associated with each identifier that represents the ranked position of that identifier in the ordered list.

Referring to FIG. 3, the computer readable medium 16, in various aspects, comprises a second digital file 30 stored thereon. The second digital file 30 comprises one or more lists 32 of microarray probe set IDs (or other identifier) associated with one or more gene expression signatures associated with rhinovirus infection or uninfected reference profiles. The listing 32 of microarray probe set IDs optionally comprises a smaller list of probe set IDs than the gene expression profile(s) of the first digital file 20. In some embodiments, the list comprises between about 2 and about 10,000 probe set IDs. In other embodiments the list comprises about 10 or more, about 50 or more, about 100 or more, about 200 or more, or about 300 or more probe set IDs. Optionally, the list comprises less than about 10,000, less than about 5,000, less than about 1,000, less than about 800, less than about 600, or less than about 400 probe set IDs. The listing 32 of probe set IDs of the second digital file 30 comprises a list of probe set IDs representing up- and/or down-regulated expression transcripts selected to represent rhinovirus infection (or a non-infected reference profile). In some embodiments, a first list represents the up-regulated transcripts (i.e., expression transcripts present in a sample at increased levels) and a second list represents the down-regulated transcripts (i.e., expression transcripts present in a sample at decreased levels) of the gene expression profile. The listing(s) are stored, e.g., in a data structure of the digital file 30 and the data arranged so that, when the digital file is read by the software application 28, a plurality of character strings are reproduced representing the list of probe set IDs. Instead of probe set IDs, equivalent gene names and/or gene symbols (or another nomenclature) are optionally substituted for a list of probe set IDs. Additional data may be stored with the gene expression signature and/or the digital file 30 and this is commonly referred to as metadata, which may include any associated information, for example, sample source and microarray identification. A plurality of gene expression signatures may be stored in a plurality of digital files and/or stored on a plurality of computer readable media, or stored in the same digital file (e.g., 30) or stored in the same digital file or database that comprises the gene expression profiles 22, 24, and 26.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices. In various embodiments, expression levels of genes in the panel are determined and compared to, e.g., reference gene expression profiles representative of rhinovirus infection or representative of the absence of rhinovirus infection or an index value or baseline value. A reference gene expression profile can also comprise expression data derived from prediction algorithms or computed indices.

The computer readable medium 16 (or another computer readable media, such as 16), in various aspects of the invention, have stored thereon one or more digital files 28 comprising computer readable instructions or software for reading, writing to, or otherwise managing and/or accessing the digital files 20, 30. The computer readable medium 16 also optionally comprises software or computer readable and/or executable instructions that cause the computing device 12 to perform one or more steps of the methods described herein including, for example and without limitation, the step(s) associated with comparing a gene expression signature stored in digital file 30 to a gene expression profile 22 stored in digital file 20. In various embodiments, the one or more digital files 28 form part of a database management system for managing the digital files 20, 30. Non-limiting examples of database management systems are described in U.S. Pat. Nos. 4,967,341 and 5,297,279.

In various aspects, the computer readable medium 16 forms part of or otherwise be connected to the computing device 12. The computing device 12 can be provided in a wide variety of forms, including but not limited to any general or special purpose computer such as a server, a desktop computer, a laptop computer, a tower computer, a microcomputer, a mini computer, and a mainframe computer.

### Screening Compounds

The gene expression profiles described herein are useful for identifying agents for improving and/or maintaining respiratory health, as well as evaluating candidate agents for activity against rhinovirus or ameliorating symptoms associated with rhinovirus infection. Indeed, the materials and methods of the invention lend themselves to screening tens to hundreds of thousands of candidate active agents in silico to identify lead candidates for further evaluation using, e.g., the methods described herein. In this regard, the invention includes systems and methods utilizing connectivity mapping to predict effectiveness of potential active agents against rhinovirus infection, symptoms of rhinovirus infection, or improvement or maintenance of respiratory health. Connectivity mapping (C-map) discovers functional connections between gene expression associated with a phenotype and cellular responses to agents. Connectivity mapping is described in detail herein and further described in, e.g., Hughes et al., Cell, 102, 109-126 (2000); and Lamb et al., Science, 313, 1929-35 (2006).

The invention includes a method of formulating a composition for treating rhinovirus infection or a symptom thereof. The method comprises accessing a plurality of instances stored on at least one computer readable medium. Each instance is associated with an agent, and each instance comprises an ordered list comprising a plurality of identifiers representing gene expression transcripts encoded by (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11. The method further comprises comparing a rhinovirus infection-associated gene expression profile to the plurality of the instances. The rhinovirus infection-associated gene expression profile comprises identifiers representing gene expression transcripts encoded by (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, and CXCL10. The comparison comprises comparing each identifier in the rhinovirus infection-associated gene expression signature with the position of the same identifier in the ordered lists for each of the plurality of instances. The method further comprises assigning a connectivity score to each of the plurality of instances; and formulating a composition comprising an agent associated with an instance having a negative connectivity score.

An exemplary system suitable for identifying an agent that treats or prevents rhinovirus or a symptom thereof comprises (a) at least one computer readable medium having stored thereon a plurality of instances, and a rhinovirus infection-associated gene expression signature, wherein each instance comprises an instance list of rank-ordered identifiers representing gene expression transcripts encoded by (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, and CXCL10. Additionally, the system comprises (b) a programmable computer comprising computer-readable instructions that cause the programmable computer to execute one or more of the following: (i) accessing the plurality of instances and the rhinovirus infection-associated gene expression signature stored on the computer medium; (ii) comparing the rhinovirus infection-associated gene expression signature to the plurality of the instances, wherein the comparison comprises comparing the each identifier in the gene expression signature list with the position of the same identifier in the instance list for each of the plurality of instances; and (iii) assigning a connectivity score to each of the plurality of instances. If desired, the system further comprises (c) a microarray scanner for receiving a sample of biomarkers; and a second programmable computer for transmitting gene expression data from the scanner to the first programmable computer.

An exemplary system is illustrated in FIG. 3, wherein a computer readable medium 16, which may be provided as a hard disk drive, comprises a digital file 20, such as a database file, optionally comprising one or more instances 22, 24, and 26 stored in a data structure associated with the digital file 20. The data stored in digital files are, in various aspects, stored in a wide variety of data structures and/or formats. The data is stored, for example, in one or more searchable databases, such as free databases, commercial databases, or a company's internal proprietary database. The database may be provided or structured according to any model known in the art such as, for example and without limitation, a flat model, a hierarchical model, a network model, a relational model, a dimensional model, or an object-oriented model. In some embodiments, at least one searchable database is a company's internal proprietary database. A user of the system may use a graphical user interface associated with a database management system to access and retrieve data from the one or more databases or other data sources to which the system is operably connected. In some embodiments, the first digital file is provided in the form of a first database and the second digital file is provided in the form of a second database. In other embodiments, the first and second digital files may be combined and provided in the form of a single file.

In some embodiments, the first digital file 20 may include data that is transmitted across the communication network 18 from a digital file 36 stored on the computer readable medium 38. In one embodiment, the first digital file 20 may comprise rhinovirus infection-associated gene expression data or instances, as well as data from the digital file 36, such as gene expression data from generated from other infections, gene expression signatures, candidate agent information, clinical trial data, scientific literature, chemical databases, and/or pharmaceutical databases. The digital file 36 is optionally provided in the form of a database, including but not limited to Sigma-Aldrich LOPAC collection, Broad Institute C-MAP collection, GEO collection, and Chemical Abstracts Service (CAS) databases.

The term “instance,” as used herein, refers to data from a gene expression profiling experiment in which cells (e.g., nasal epithelial cells) are dosed with a candidate agent. In some embodiments, the data comprises a list of identifiers representing the genes that are part of the gene expression profiling experiment. The identifiers include, e.g., gene names, gene symbols, microarray probe set IDs, or any other identifier. An instance comprises, in various circumstances, data from a microarray experiment and comprises a list of probe set IDs of the microarray ordered by the extent of differential expression relative to a control. The data may also comprise metadata, including but not limited to data relating to one or more candidate agents, the gene expression profiling test conditions, the cells, and the microarray. Numerous instances are publicly available via an on-going, large scale community C-map project, which is available under the “supporting materials” hyperlink at http://www.sciencemag.org/content/313/5795/1929/suppl/DC1.

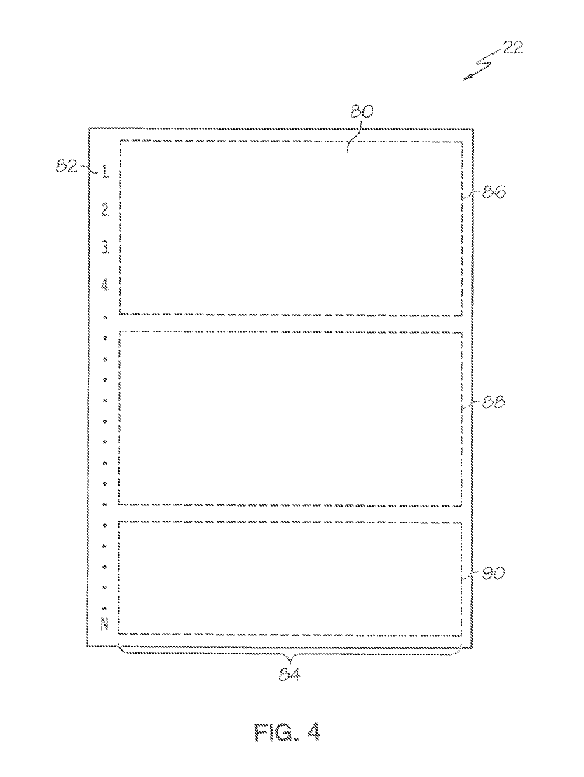

In various embodiments, the probe sets are rank ordered by the fold change relative to the controls in the same C-map batch (single instance/average of controls) to reflect the most up-regulated to the most down-regulated. For example, the probes are sorted into a list according to the level of gene expression regulation detected, wherein the list progresses from high levels of expression to marginal or no alteration in expression levels to decreased levels of expression. The rank ordered listing of probe IDs is stored as an instance. Referring to FIG. 4, the data associated with an instance comprises the probe ID 80 and a value 82 representing its ranking in the list (e.g., 1, 2, 3, 4 . . . N, where N represents the total number of probes on the microarray). The ordered list 84 may generally comprise approximately three groupings of probe IDs: a first grouping 86 of probe IDs associated with up-regulated expression transcripts, a second group 88 of probe IDs associated with transcripts demonstrating marginal increase or decrease or no detectable signal or noise, and a third group 90 of probe IDs associated with reduced expression. The most up-regulated transcripts are at or near the top of the list 84 and the most down-regulated transcripts are at or near the bottom of the list 84. The groupings are shown for illustration, but the lists for each instance may be continuous and the number of regulated genes will depend on the strength of the effect of the agent associated with the instance. Other arrangements within the list 84 are contemplated. For example, the probe IDs associated with the down-regulated genes may be arranged at the top of the list 84. This instance data may also further comprise metadata such as agent identification, agent concentration, cell line or sample source, and microarray identification.

In some embodiments, one or more instances comprise at least about 1,000, 2,500, 5,000, 10,000, or 20,000 identifiers and/or less than about 30,000, 25,000, or 20,000 identifiers. In some embodiments, the database comprises at least about 50, 100, 250, 500, or 1,000 instances and/or less than about 50,000, 20,000, 15,000, 10,000, 7,500, 5,000, or 2,500 instances (e.g., between about 50-50,000 instances or between about 1,000-2,000 instances).

Any substance, chemical, compound, active, natural product, extract, drug (e.g., Sigma-Aldrich LOPAC (Library of Pharmacologically Active Compounds) collection), small molecule, peptide, protein, peptidomimetic, polynucleotide or combination of any of the foregoing is a suitable “agent” in the context of the inventive method. There are a number of different libraries used for the identification of agents, including, but not limited to, chemical libraries, natural product libraries, and combinatorial libraries comprising peptides and/or organic molecules. A chemical library, in some aspects, consists of structural analogs of known compounds or compounds that are identified as “hits” or “leads” via other screening methods. Natural product libraries are collections of substances isolated from or produced by microorganisms, animals, plants, or marine organisms. Combinatorial libraries are composed of large numbers of peptides or organic compounds.

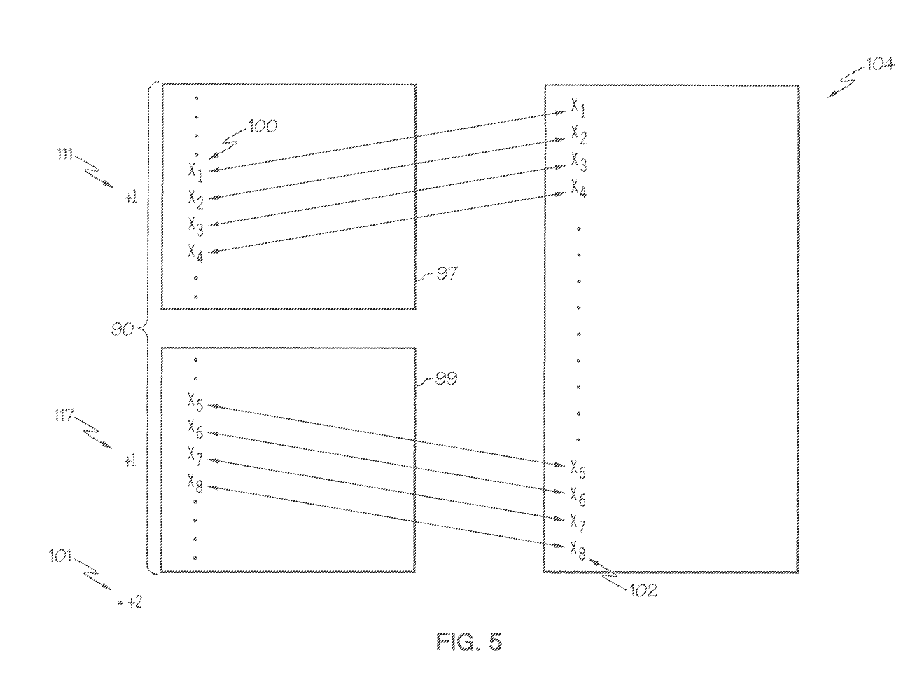

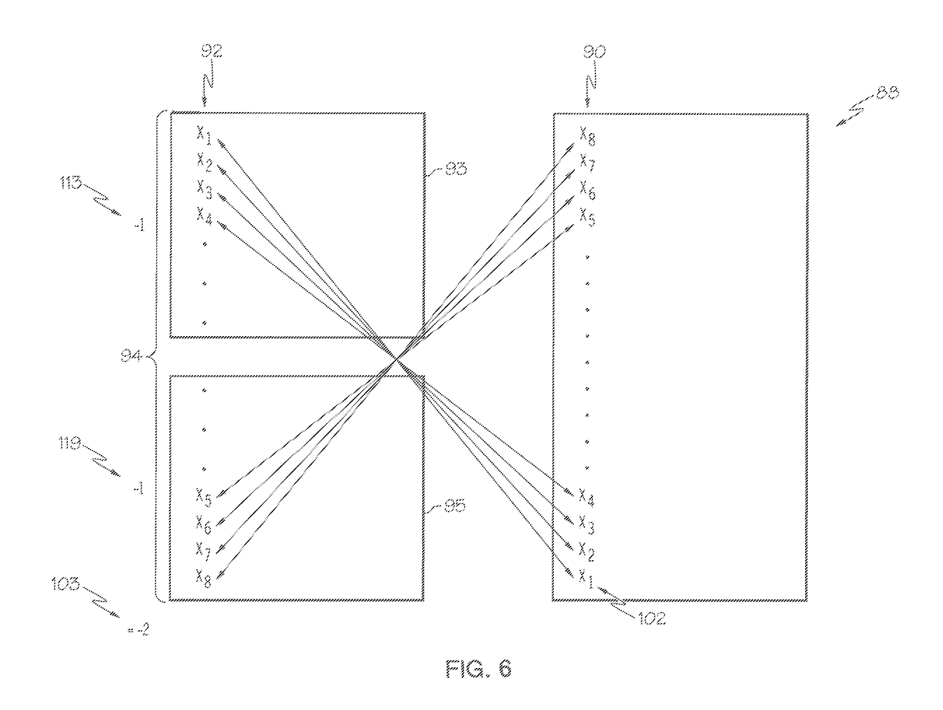

The “connectivity score” is generated by applying a statistical method to determine how strongly the rhinovirus infection-associated gene expression signature matches the gene expression signature of an instance. Positive connectivity occurs when, e.g., the expression transcripts in an up-regulated signature from rhinovirus infection are also enriched among the up-regulated expression transcripts in an instance, and the expression transcripts in a down-regulated signature from rhinovirus infection are also enriched among the down-regulated expression transcripts in an instance. On the other hand, if the up-regulated expression transcripts of the signature are predominantly found among the down-regulated expression transcripts of the instance, and vice versa, this is scored as “negative connectivity.” FIG. 5 schematically illustrates an extreme example of a positive connectivity between signature 90 and the instance 104 comprising the probe IDs 102, wherein the probe IDs of the instance are ordered from most up-regulated to most down-regulated. In this example, the probe IDs 100 (e.g., X1, X2, X3, X4, X5, X6, X7, X8) of the gene expression signature 90, comprising an up list 97 and a down list 99, have a one to one positive correspondence with the most up-regulated and down regulated probe IDs 102 of the instance 104, respectively. Similarly, FIG. 6 schematically illustrates an extreme example of a negative connectivity between signature 94 and the instance 88 comprising the probe IDs 90, wherein the probe IDs of the instance are ordered from most up-regulated to most down-regulated. In this example, the probe IDs of the up list 93 (e.g., X1, X2, X3, X4) correspond exactly with the most down-regulated genes of the instance 88, and the probe IDs of the down list 95 (e.g., X5, X6, X7, X8) correspond exactly to the most up-regulated probe IDs of the instance 88.

In some embodiments, the connectivity score can be a combination of an up-score and a down-score, wherein the up-score represents the correlation between the up-regulated expression transcripts of a gene expression signature and an instance and the down-score represents the correlation between the down-regulated transcripts of a gene expression signature and an instance. The up-score and down-score have values between, e.g., +1 and −1. For an up-score, a high positive value indicates that the corresponding agent of an instance induced the expression of transcripts bound by microarray probes that bind the transcripts of the up-regulated genes of the gene signature, and a high negative value indicates that the corresponding agent associated with the instance repressed the expression of the transcript bound by microarray probes of the up-regulated transcript of the rhinovirus infection-associated gene signature. The up-score is calculated by comparing each identifier of an up list of a gene signature comprising the up-regulated transcripts (e.g., lists 93, 97, and 107) to an ordered instance list. The down-score is calculated by comparing each identifier of a down list of a gene signature comprising the down-regulated genes (see, e.g., lists 95, 99, and 109) to an ordered instance list. In these exemplary embodiments, the gene signature comprises the combination of the up list and the down list.

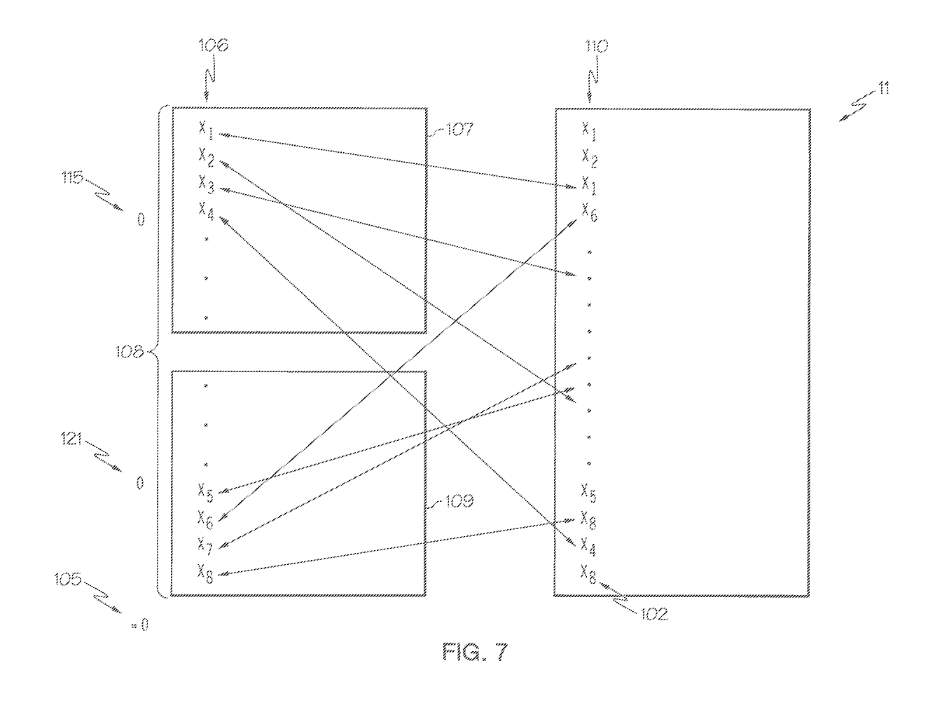

In some embodiments, the connectivity score value may range from +2 (greatest positive connectivity) to −2 (greatest negative connectivity), wherein the connectivity score (e.g., 101, 103, and 105) is the combination of the up score (e.g., 111, 113, 115) and the down score (e.g., 117, 119, 121) derived by comparing each identifier of a gene signature to the identifiers of an ordered instance list. In other embodiments the connectivity range may be between +1 and −1. Examples of the scores are illustrated in FIGS. 5-7 as reference numerals 101, 103, 105, 111, 113, 115, 117, 119, and 121. The strength of matching between a profile and an instance represented by the up scores and down scores and/or the connectivity score may be derived by one or more approaches known in the art and include, but are not limited to, parametric and non-parametric approaches. Examples of parametric approaches include Pearson correlation (or Pearson r) and cosine correlation. Examples of non-parametric approaches include Speannan's Rank (or rank-order) correlation, Kendall's Tau correlation, and the Gamma statistic. Generally, in order to eliminate a requirement that all profiles be generated on the same microarray platform, a non-parametric, rank-based pattern matching strategy based on the Kolmogorov-Smirnov statistic is used (see M. Hollander et al. “Nonparametric Statistical Methods”; Wiley, New York, ed. 2, 1999)(see, e.g., pp. 178-185). It will be appreciated that where all expression signatures are derived from a single technology platform, similar results may be obtained using conventional measures of correlation, for example, the Pearson correlation coefficient.

In specific embodiments, the methods and systems of the invention employ the nonparametric, rank-based pattern-matching strategy based on the Kolmogorov-Smirnov statistic, commonly known as Gene Set Enrichment Analysis (GSEA) (see, e.g., Subramanian, A. et al. Proc. Natl. Acad. Sci. U.S.A, 102, 15545-15550 (2005)). For each instance, a down score is calculated to reflect the match between the down-regulated genes of the query and the instance, and an up score is calculated to reflect the correlation between the up-regulated genes of the query and the instance. In certain embodiments the down score and up score each may range between −1 and +1. The combination represents the strength of the overall match between the query signature and the instance.

The combination of the up score and down score is used to calculate an overall connectivity score for each instance, and in embodiments where up and down score ranges are set between −1 and +1, the connectivity score ranges from −2 to +2, and represents the strength of match between a query signature and the instance. The sign of the overall score is determined by whether the instance links positivity or negatively to the signature. Positive connectivity occurs when the agent associated with an instance tends to up-regulate the expression transcripts in the up list of the signature and down-regulate the expression transcripts in the down list. Conversely, negative connectivity occurs when the agent reverses the up- and down-signature gene expression changes. The magnitude of the connectivity score is the sum of the absolute values of the up and down scores when the up and down scores have different signs. A high positive connectivity score predicts that the agent will tend to induce the condition that was used to generate the query signature, and a high negative connectivity score predicts that the agent will reverse the condition associated with the query signature. A zero score is assigned where the up- and down-scores have the same sign, indicating that an agent did not have a consistent impact the condition signature (e.g., up-regulating both the up and down lists).

Each instance is, in various aspects, rank-ordered according to its connectivity score to the query signature and the resulting rank-ordered list displayed to a user using any suitable software and computer hardware allowing for visualization of data.

In various embodiments, the methods may comprise identifying from the displayed rank-ordered list of instances (i) one or more agents associated with the instances of interest (thereby correlating activation or inhibition of a plurality of expression transcripts listed in the query signature to the one or more agents); (ii) the differentially expressed transcripts associated with any instances of interest (thereby correlating the transcripts with the one or more agents); or (iii) a combination thereof. The one or more agents associated with an instance is identified, e.g., from metadata stored in the database for that instance. It will be appreciated, however, that agent data for an instance may be retrievably stored in and by other means. Because the identified agents statistically correlate to increased or decreased levels of biomarkers (e.g., mRNA or cDNA) listed in the query profile, and because the query profile is a proxy for rhinovirus infection, the identified agents are candidates for new formulations for treating rhinovirus infection and/or maintaining or improving respiratory health.

In some embodiments, the methods of the invention may further comprise testing a selected candidate agent, using in vitro assays and/or in vivo testing, to validate the activity of the agent and usefulness in a formulation for treating rhinovirus infection, ameliorating one or more rhinovirus infection symptoms, or maintaining or improving respiratory health. Any suitable in vitro test method can be used, including those known in the art, and most preferably in vitro models having an established nexus to the desired in vivo result. In some embodiments, evaluation of selected agents using in vitro assays reveal and/or confirm that one or more new candidate agents are suitable for use in conjunction with a known actives that improve or maintain respiratory health.

### Formulations and Method of Maintaining or Improving Respiratory Health

The invention further provides a composition comprising an agent useful in maintaining or improving respiratory health and well being, such as an agent identified by the methods described herein. In one aspect, the composition is a physiologically-acceptable composition, such as a pharmaceutical composition, comprising one or more agents demonstrating an inverse gene expression signature (i.e., negative connectivity score) compared to a rhinovirus infection-associated gene expression signature. Exemplary agents include, but are not limited to, splitomicin, phenyl biguanide, beclometasone, tomelukast, flupentixol, decitabine, topiramate, tiabendazole, pirlindole, pancuronium bromide, metaraminol, yohimbic acid, arachidonic acid, myosmine, proscillaridin, naftidrofuryl, terconazole, sulfaphenazole, pheniramine, eldeline, atropine, ethonitrate, digitoxigenin, fludroxycortide, diflorasone, tremorine, 3-acetamidocoumarin, benzocaine, mevalolactone, harmol, sparteine, digoxigenin, tacrolimus, lomustine, fipexide, lidoflazine, alfaxalone, suramin sodium, chenodeoxycholic acid, meticrane, etidronic acid, oxymetazoline, amiprilose, gibberellic acid, helveticoside, lanatoside C, chloramphenicol, cefotiam, diphemanil metilsulfate, guanethidine, fursultiamine, pivmecillinam, letrozole, memantine, allantoin, etacrynic acid, raubasine, 16-phenyltetranorprostaglandin E2, etomidate, moracizine, lidocaine, ciclosporin, fluticasone, ofloxacin, droperidol, piromidic acid, lisinopril, atovaquone, levodopa, ajmaline, velnacrine, doxorubicin, acebutolol, tocamide, sulpiride, furaltadone, pentamidine, iobenguane, guaifenesin, oleandomycin, dizocilpine, bezafibrate, flumetasone, nitrofural, methyldopate, stachydrine, doxepin, amantadine, midodrine, biperiden, monensin, clebopride, 16,16-dimethylprostaglandin E2, eucatropine, niridazole, tridihexethyl, harpagoside, palmatine, dicloxacillin, pronetalol, triamcinolone, gemfibrozil, ronidazole, deptropine, raloxifene, sulfamethoxazole, 6-bromoindirubin-3′-oxime, fusidic acid, olivem 450, dimethyl caffeic acid, citrus extract, pyrogallol, norcamphor, raspberry ketone, omega-pentadecalactone, enoxolone, usnic acid, tartaric acid, resveratrol, Peppermint oil, vanillin, rosmarinic acid, silymarin, emulphor, neem oil, caffeine, taurine, D-carvone, cis-jasmone, rice bran oil, chrysin, thymol, lauric diethanolamide, FD&C Green No. 3, thioglycerol, anethole, sorbitan sesquioleate, stearyl citrate, meglumine, carmine, mevalolactone, ethacrynic acid, velnacrine, benzafibrate, methyldopa, glycitein, octyl gallate, lauryl gallate, pinocembrin, and apigenin. Exemplary agents also include, but are not limited to, ammonium lauryl sulfate, n-lauroylsarcosine, dextran, gamma-cyclodextrin, sorbitan monooleate, squalene, aluminum stearate, and lupulin extract (hops). Exemplary agents further include, but are not limited to, octoxynol-1,2-amino-2-methyl-1-propanol, dichlorobenzyl alcohol, glutamic acid hydrochloride, octanoic acid, cupric sulfate, deoxycholic acid, PVM/MA copolymer, pyrogallol, usnic acid, fisetin, quercetin, scuttelarein, epicatechin 3-gallate, galic acid, myricetin, kaempferol, epigallocatechin, epigallocatechin 3-gallate, cyanidin, delphinidin, NDGA, propyl gallate, and conjugated linoleic acid. In one aspect, the composition comprises one or more of the following: olivem 450, enoxolone, silymarin, rosemarinic acid, gum rosin, trideceth-10, and isoeugenol.

In some variations, the agent(s) is an excipient combined with one or more other agents for maintaining or improving respiratory health. For example, the invention includes a composition comprising (i) a therapeutic agent and (ii) an excipient selected from the group consisting of olivem 450, usnic acid, silymarin, gum rosin, trideceth-10, and combinations thereof (e.g., olivem 450, silymarin, gum rosin, trideceth-10, or a combination of any of the foregoing). In one aspect, the therapeutic agent is selected from the group consisting of an antihistamine, an antitussive, a decongestant, an expectorant, and combinations thereof.

Any pharmaceutically-acceptable antihistamine, antitus sive, decongestant, and/or expectorant can be used in the context of the invention. Exemplary antihistamines include, but are not limited to, fexofenadine, chlorpheniramine, desloratadine, levocetirizine, diphenhydramine, doxylamine succinate, triprolidine, clemastine, pheniramine, brompheniramine, dexbrompheniramine, loratadine, cetirizine, fexofenadine, amlexanox, alkylamine derivatives, cromolyn, acrivastine, ibudilast, bamipine, ketotifen, levocetirizine, nedocromil, omalizumab, dimethindene, oxatomide, pemirolast, pyrrobutamine, pentigetide, thenaldine, picumast, tolpropamine, ramatroban, repirinast, suplatast tosylate aminoalkylethers, tazanolast, bromodiphenhydramine, tranilast, carbinoxamine, traxanox, chlorphenoxamine, diphenylpyaline, embramine, p-methyldiphenhydramine, moxastine, orphenadrine, phenyltoloxamine, setastine, ethylenediamine derivatives, chloropyramine, chlorothen, methapyrilene, pyrilamine, talastine, thenyldiamine, thonzylamine hydrochloride, tripelennamine, piperazines, chlorcyclizine, clocinizine, homochlorcyclizine, hydroxyzine, tricyclics, phenothiazines, mequitazine, promethazine, thiazinamium methylsulfate, azatadine, cyproheptadine, deptropine, isothipendyl, olopatadine, rupatadine, antazoline, astemizole, azelastine, bepotastine, clemizole, ebastine, emedastine, epinastine, levocabastine, mebhydroline, mizolastine, phenindamine, terfenadine, tritoqualine, and combinations thereof. Examples of antitussives (cough suppressants) include, e.g., dextromethorphan, menthol, codeine, chlophedianol, levodropropizine, and combinations thereof. Decongestants are known in the art and include, for example, pseudoephedrine, pseudoephedrine hydrochloride, phenylephrine, phenylephrine hydrochloride, phenylpropanolamine, oxymetazoline, xylometazoline, naphazoline, 1-desoxyephedrine, ephedrine, propylhexedrine, and combinations thereof. Exemplary expectorants include, but are not limited to, guaifenesin, ambroxol, bromhexine, and combinations thereof. The composition may include any pharmaceutically acceptable salts, metabolites, and combinations of the above-listed actives.

Alternatively or in addition, the composition comprises a therapeutic agent selected from the group consisting of an analgesic, an anticholinergic, an anti-inflammatory, an antipyretic, an antiviral, a demulcent, a mucolytic, an anesthetic, and combinations thereof. In various embodiments, the analgesic, anti-inflammatory or antipyretic is acetaminophen or a nonsteroidal anti-inflammatory drug (NSAID), such as, e.g., choline and magnesium salicylates, choline salicylate, celecoxib, diclofenac potassium, diclofenac (e.g., diclofenac sodium optionally with misoprostol), diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, valdecoxib, or a combination of any of the foregoing, as well as prescription analgesics, non-limiting examples of which include propyxhene HCl, codeine, mepridine, and combinations thereof. Antivirals include, but are not limited to, amantidine, rimantadine, pleconaril, zanamivir, oseltamivir, and combinations thereof.

The mucolytic is optionally selected from the group consisting of ambroxol, N-acetylcysteine, bromhexine and combinations thereof. Non-limiting examples of anticholinergics include ipratropium, chlorpheniramine, brompheniramine, diphenhydramine, doxylamine, clemastine, triprolidine, and combinations thereof. Non-limiting examples of demulcents include glycerin, honey, pectin, gelatin, slippery elm bark, liquid sugar, glycyrrhizin (licorice), and combinations thereof. Anesthetics include, but are not limited to, phenol, menthol, dyclonine HCl, benzocaine, lidocaine, hexylresorcinol, and combinations thereof. Examples of antibiotics include, but are not limited to, nitroimidazole antibiotics, tetracyclines, penicillin-based antibiotics such as amoxicillin, cephalosporins, carbopenems, aminoglycosides, macrolide antibiotics, lincosamide antibiotics, 4-quinolones, fluoroquinolones, rifamycins, macrolides, nitrofurantoin, and combinations thereof. Any pharmaceutically acceptable salts, metabolites, and combinations of the above-listed actives also are contemplated. In one aspect, the composition comprises the excipient(s), an analgesic, a decongestant, and an antitussive, wherein the analgesic is acetaminophen, the decongestant is phenylephrine, and the antitussive is dextromethorphan. It will be appreciated that invention includes compositions wherein the analgesic, the anticholinergic, the anti-inflammatory, the antipyretic, the antiviral, the demulcent, and/or the mucolytic replaces the antihistamine, the antitussive, the decongestant, or the expectorant in the composition described above.

In various embodiments, the composition comprises one or more additional components. Nonlimiting examples of additional components optionally included in the composition include cooling agents such as menthol, warming agents, flavoring agents, salivating agents, tea extract, vitamin(s) (e.g., Vitamin A, Vitamin C, Vitamin B, and/or Vitamin D), carotenoid, rosemary, rosemary extract, caffeic acid, coffee extract, tumeric extract, curcumin, blueberry extract, grapeseed extract, rosemaric acid, antioxidant, amino acid, enzyme, prebiotic, probiotic, andrographis extract, 1-tryptophan, Allium sativum, herbal remedies, vitamins, supplements, antioxidants, natural ingredients, minerals, energy boosting ingredients, sleep aids, immune system boosting agents, colorant, preservative, fragrance, fruit extract, and combinations thereof. When present, the composition comprises from about 10⁶ to 10¹² colony forming unit (cfu) of a probiotic, and alternatively from about 10⁶ to 10¹⁰ cfu of a probiotic. The probiotic component is, in one aspect, a lactic acid bacteria, e.g., bacteria of the genera Bacillus, Bacteroides, Bifidobacterium, Enterococcus (e.g., Enterococcus faecium), Lactobacillus, and Leuconostoc, and combinations thereof. In another embodiment of the invention, the probiotic is selected from bacteria of the genera Bifidobacterium, Lactobacillus, and combinations thereof.

In many variations of the invention, the composition comprises a pharmaceutically acceptable carrier, e.g., one or more solvents, dispersion media, coatings, antibacterial and antifungal agents, isotonic and absorption delaying agents, and the like, compatible with administration to a mammal, such as a human. Any carrier compatible with the excipient(s) and therapeutic agent(s) is suitable for use. Supplementary active compounds may also be incorporated into the compositions. A composition of the invention is formulated to be compatible with its intended route of administration. Examples of routes of administration include oral administration (ingestion) and parenteral administration, e.g., intravenous, intradermal, subcutaneous, inhalation, nasal, transdermal (topical), transmucosal, buccal, sublingual, pulmonary and rectal administration. Solutions or suspensions used for parenteral application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide.

Oral compositions generally include an inert diluent or an edible carrier. Oral formulations generally take the form of a pill, tablet, capsule (e.g., softgel capsule, solid-filled capsule, or liquid-filled capsule), solid lozenge, liquid-filled lozenge, mouth and/or throat drops or spray, effervescent tablets, orally disintegrating tablet, suspension, emulsion, syrup, elixir, or tincture. The composition may be contained in enteric forms to survive the stomach or further coated or mixed to be released in a particular region of the gastrointestinal tract by known methods. Solid oral dosage forms are typically swallowed immediately, or slowly dissolved in the mouth. Oral compositions may also be prepared using a fluid carrier for use as a mouthwash, wherein the compound in the fluid carrier is applied orally and swished and expectorated or swallowed. Oral formulations optionally contain any of the following ingredients, or compounds of a similar nature: a binder such as microcrystalline cellulose, gum tragacanth or gelatin; starch or lactose; a disintegrating agent such as alginic acid, Primogel™, or corn starch; a lubricant such as magnesium stearate; a glidant such as colloidal silicon dioxide; and/or a sweetening agent such as sucrose or saccharin.

An oral formulation optionally further comprises a flavoring agent. Nonlimiting examples include EVERCOOL 180 available from Givaudan of Cincinnati, Ohio which is available, for example, as a 5% solution of N-(4-cyanomethylphenyl)-p-menthanecarboxamide in a flavoring ingredient cool white grape, or as a 7.5% solution of N-(4-cyanomethylphenyl)-ρ-menthanecarboxamide in a flavor ingredient such as spearmint or peppermint. Other flavor ingredients include, but are not limited, to peppermint oil, corn mint oil, spearmint oil, oil of wintergreen, clove bud oil, cassia, sage, parsley oil, marjoram, lemon, lime, orange, cherry, cis-jasmone, 2,5-dimethyl-4-hydroxy-3(2H)-furanone, 5-ethyl-3-hydroxy-4-methyl-2(5H)-furanone, vanillin, ethyl vanillin, anisaldehyde, 3,4-methylenedioxybenzaldehyde, 3,4-dimethoxybenzaldehyde, 4-hydroxybenzaldehyde, 2-methoxybenzaldehyde, benzaldehyde; cinnamaldehyde, hexyl cinnamaldehyde, alpha-methyl cinnamaldehyde, ortho-methoxy cinnamaldehyde, alpha-amyl cinnamaldehyde, propenyl guaethol, heliotropine, 4-cis-heptenal, diacetyl, methyl-ρ-tert-butyl phenyl acetate, menthol, methyl salicylate, ethyl salicylate, 1-menthyl acetate, oxanone, alpha-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, alpha-terpineol, linalool, limonene, citral, maltol, ethyl maltol, anethole, dihydroanethole, carvone, menthone, β-damascenone, ionone, gamma decalactone, gamma nonalactone, gamma undecalactone and mixtures thereof.

For administration by inhalation, the composition is optionally delivered in the form of a spray. The spray may be an aerosol spray from a pressured container or dispenser, which contains a suitable propellant, e.g., a gas such as carbon dioxide, or a nebulizer. The composition is optionally formulated for delivery via a dry powder inhaler (DPI), a metered dose inhaler (pMDI), nasal spray, or a vaporizer. For routes of administration involving absorption of an agent and/or excipient through mucosal membrane, the composition further optionally comprises a penetrant.

Optionally, the composition is formulated as a “liquid respiratory composition,” i.e., a composition in a form that is deliverable to a mammal via the oral cavity, mouth, throat, nasal passage or combinations thereof. These compositions can be delivered by a delivery device selected from droppers, pump, sprayers, liquid dropper, spoon, cup, squeezable sachets, power shots, and other packaging and equipment, and combinations thereof. In one embodiment, the liquid respiratory composition comprises the therapeutic agent, and excipient, a thickening polymer (e.g., xanthan gum, cellulosic polymers such as carboxymethycellulose (CMC), hydroxethylcellulose, hydroxymethylcellulose, and hydroxypropylmethylcellulose, carrageenan, polyacrylic acid, cross-linked polyacrylic acid such as Carbopol®, polycarbophil, alginate, clay, and combinations thereof), and optionally a mucoadhesive polymer (e.g., polyvinylpyrrolidone (Povidone), methyl vinyl ether copolymer of maleic anhydride (Gantrez®), guar gum, gum tragacanth, polydextrose, cationic polymers, poly(ethylene oxide), poly(ethylene glycol), poly(vinyl alcohol), poly(acrylic acid), cross-linked polyacrylic acid such as Carbopol®, polycarbophil, poly(hydroxylethyl methacrylate), chitosan, cellulosic polymers such as carboxymethycellulose (CMC), hydroxethylcellulose, hydroxymethylcellulose, and hydroxypropylmethylcellulose, and combinations thereof). The composition is preferably a non-Newtonian liquid that exhibits zero shear viscosity from about 100 centiPoise (cP) to about 1,000,000 cP, from about 100 cP to about 500,000 cP, from about 100 cP to about 100,000 cP, from about 100 cP to about 50,000 cP, from about 200 cP to about 20,000 cP, from about 1,000 to about 10,000 cP at a temperature of about 37° C., as measured according to the Shear Viscosity Method. The pH range of the formulation is generally from about 1 to about 7, from about 2 to about 6.5, and from about 4 to about 6.

In various embodiments, in addition to the excipient(s) and therapeutic agent(s) described herein, a nasal spray formulation comprises benzalkonium chloride, camphor, chlorhexidine gluconate, citric acid, disodium EDTA, eucalyptol, menthol, purified water, and/or tyloxapol. An exemplary oral composition comprises FD&C Blue No. 1, gelatin, glycerin, polyethylene glycol, povidone, propylene glycol, purified water, sorbitol special, and/or titanium dioxide in addition to an excipient and acetaminophen, doxylamine succinate, and phenylephrine HCl (or dextromethorphan).

Pharmaceutical compositions suitable for injectable use include sterile aqueous solutions (where water-soluble), or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition is sterile and fluid to allow syringability. The carrier may be a solvent or dispersion medium containing, for example, water, ethanol, polyol (for example, glycerol, propylene glycol, and liquid polyethylene glycol, and the like), and suitable mixtures thereof. Fluidity is maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin. The injectable preparations may be enclosed in ampules, disposable syringes or multiple dose vials made of glass or plastic.

In one embodiment, the components of the composition are prepared with carriers that will protect the components against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid.

The formulation is provided, in various aspects, in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms are dictated by and are directly dependent on the unique characteristics of the excipient(s) and therapeutic agent(s) and the particular biological effect to be achieved.

The invention further includes a method of maintaining or improving respiratory health using any of the compositions described herein. For example, in one embodiment, the method comprises administering to a subject a composition comprising an excipient selected from the group consisting of olivem 450, usnic acid, silymarin, gum rosin, or trideceth-10 (or combinations thereof), optionally further comprising an analgesic, an anticholinergic, an antihistamine, an anti-inflammatory, an antipyretic, an antitussive, an antiviral, a decongestant, a demulcent, an expectorant, a mucolytic, or combination of any of the foregoing. Analgesics, anticholinergics, antihistamines, anti-inflammatories, antipyretics, antitussives, antivirals, decongestants, demulcents, expectorants, and mucolytics are described above. In one embodiment, the composition comprises olivem 450, usnic acid, silymarin, gum rosin, and/or trideceth-10 in combination with acetaminophen, phenylephrine, and dextromethorphan. The composition, in various aspects, further comprises one or more of the additional components described herein. Optionally, the composition is administered to the subject orally in an amount sufficient to maintain or improve respiratory health of the subject.

In various aspects of the inventive method, “improving respiratory health” comprises reduced sneezing, reduced nasal discharge, reduced swelling of mucosal membranes, reduced wheezing, reduced hoarseness, reduced coughing, and reduced sinusitis. The subject may be suffering from a respiratory ailment or distress (e.g., allergies, asthma, bronchitis, the symptoms of rhinovirus infection, other viral or bacterial infection (such as pneumonia), pleural cavity ailments (such as emphysema), shortness of breath, and the like). Alternatively, the subject is not suffering from a respiratory ailment, and the composition is administered as a preventative measure to maintain respiratory health.

Safety and efficacy of compositions described herein are determined by standard procedures using in vitro or in vivo technologies, such as the materials and methods described herein. Administration may be on an as-needed or as-desired basis, for example, once-monthly, once-weekly, or daily, including multiple times daily, for example, at least once daily, from one to about ten times daily, from about two to about four times daily, or about three times daily. A dose of composition optionally comprises about from about 0.001 mg to about 1000 mg active agent, alternatively from about 2.5 mg to about 750 mg active agent, and alternatively from about 5 mg to about 650 mg of the active agent. A dose of composition optionally comprises about from about 0.001 mg to about 1000 mg excipient, alternatively from about 2.5 mg to about 750 mg excipient, and alternatively from about 5 mg to about 650 mg of the excipient.

The invention is further described in the following examples. The example serves only to illustrate the invention and are not intended to limit the scope of the invention in any way.

### EXAMPLES

### Example 1

Gene expression was assessed by microarray analysis of polynucleotides isolated from nasal scrapings obtained from subjects before and during experimental infections with human rhinovirus 16 (HRV-16) as described in Proud et al., Am. J. Respir. Crit. Care Med., 178, 962-968 (2008). Briefly, one group of individuals was inoculated with HRV-16, and a control group of individuals were shaminoculated. Nasal epithelial scrapings were collected from alternating nostrils 14 days prior to inoculation, eight hours post-innoculation, and 48 hours post-innoculation. Total RNA was extracted from nasal scrapings with TRIzol reagent (Invitrogen) and purified using RNeasy columns (Qiagen). RNA was quantitated using microarray chips (Affymetrix) comprising probes for more than 47,000 transcripts.

Analysis of covariance (ANCOVA) statistical analysis, a cut-off based on fold change in expression, and weighted voting algorithms with a cross model validation model were used to identify a high quality gene panel that is unique and representative for rhinovirus infection. Over 54,613 gene transcripts from samples were analyzed using Affymetrix microarray chips from samples taken from a control group and a group exposed to rhinovirus 16 (RV16) prior to inoculation (“baseline”), eight hours post-inoculation, and 48 hours post inoculation. Levels of gene transcript expression at baseline were compared between groups using ANOVA. ANCOVA, with main effects for gender and group, was used to compare data for each post-inoculation time point. The Wilcoxon Matched-pairs Signed-ranks test was used for nonparametric data.

At p-value<0.05, 1862 gene transcripts demonstrated significant alternations in expression levels between the RV16 group and the control group at eight hours post-inoculation. At 48 hours post-inoculation, 11,887 gene transcripts demonstrated significant alterations in expression levels between the control and RV16 groups. Because the expression of only a limited number of transcripts was significantly changed at eight hours post-inoculation (less than the number expected to be significant at p-0.05 by chance alone), the preliminary panel of transcripts from samples taken 48 hours post-inoculation was used for further analysis to generate a gene panel.

The expression levels of the 11,887 gene transcripts were quantitated and those transcripts from the RV16 group that were elevated more than two fold or decreased by more than half compared to the control group were designated as significant. The “fold change cut-off” of 2 (for up-regulation) or 0.5 (for down-regulation) reduced the panel to 688 gene transcripts.

Next, a weighted voting algorithm was applied to the 688 gene transcript set. The weighted voting method makes a weighted linear combination of relevant “signature” features obtained in the training set to provide a classification scheme for new samples. Target classes (Class 1: the RV16 group; Class 0: the control group) are known, defined parameters for a training set defined by experimental intervention and validated by symptom assessment. Data from 32 validated samples were used as a training set. The selection of classifier features (“signature” features) was accomplished by computing a signal-to-noise statistic Sx=(μ0−μ1)/(σ0+σ1), where μ0 is the mean of Class 0 and σ0 is the standard deviation of Class 0, or by reading in the list of input transcript features (i.e., the 688 selected transcript genes, described in previous steps). The class predictor was uniquely defined by the initial set of samples and markers. A set of 100 class predictors, representing a panel of 82 genes, was identified as a class predictor (“signature” or “profile”) for RV16 infection.

In addition to computing Sx to identify signature features of rhinovirus infection, the model was used to test and validate the gene panel using a weighted voting cross validation algorithm. The algorithm finds the decision boundaries between the class means: Bx=(μ0+μ1)/2, for each feature x. To predict the class (infected or not infected) of a test sample “y,” each feature “x” in the feature set casts a vote: Vx=Sx (Gxy−Bx), and the final vote for Class 0 (not infected) or Claims 1 (infected) is sign(Sx Vx). The strength or confidence in the prediction of the winning class is (Vwin−Vlose)/(Vwin+Vlose) (i.e., the relative margin of victory for the vote). The validation result showed that the panel of 82 genes has a prediction power of 85%. The panel of 82 genes is listed in Table 1. Twenty-six of the 82 genes are provided (fold-change of expression levels at 48 hours post infection provided in parentheses): RNFT2 (0.21), CRY2 (0.28), C10ORF95 (0.296), BTG4 (0.302), PSD3 (0.31), CAPN9 (0.415), SULT1E1 (0.44), HEY1 (0.443), LRRC36 (0.454), RAB3B (0.47), ALDH3B1 (0.486), FAM134B (0.486), B3GAT3 (0.493), FAS (2.08), PLSCR1 (2.53), CLEC2B (2.64), BATF3 (2.71), HAS2 (3.25), MX1 (3.29), SP110 (4.03), GBP1 (5.82), IFIT3 (7.56), IFIT1 (8.82), CXCL9 (12.8), CXCL10 (25.5), and CXCL11 (37.4). Several of the gene products can be classified according to biological activity such as, for example, cell cycle arrest (e.g., BTG4), inflammatory response (e.g., CLEC2B, CXCL11, CXCL9, and CXCL10), interferon signaling pathway (e.g., GBP1, IFIT1, and IFIT3), hyaluronan biosynthesis (e.g., HAS2), metabolic-glycosaminoglycan biosynthetic process (e.g., B3GAT3), metabolic-oxidation reduction (e.g., ALDH3B1), metabolic-steroid metabolic process (e.g., SULT1E1), protein transport (e.g., RAB3B), proteome degradation (e.g., FAS, CAPN9, and PSD3), transcription regulations (e.g., SP110, BATF3, CRY2, and HEY1), viral response (e.g., MX1 and PLSCR1), and pain perception (e.g., FAM134B).

### Example 2

This example describes a method of using a gene panel for human rhinovirus infection and a pattern matching approach to identify candidate agents for maintaining or improving respiratory health.

The gene panel identified in Example 1 was used to query databases comprising gene expression profile data obtained from cells exposed to chemical compounds to identify compounds that share a reverse expression pattern (i.e., a “negative connectivity”) with the gene expression profile determined from rhinovirus infected patients.

The following compounds were identified (connectivity score provided in parentheses): fisetin (−0.968), splitomicin (−0.926), phenyl biguanide (−0.924), beclometasone (−0.905), tomelukast (−0.901), flupentixol (−0.874), decitabine (−0.862), topiramate (−0.855), tiabendazole (−0.819), pirlindole (−0.818), pancuronium bromide (−0.801), metaraminol (−0.787), yohimbic acid (−0.783), arachidonic acid (−0.748), myosmine (−0.74), proscillaridin (−0.736), naftidrofuryl (−0.734), terconazole (−0.72), sulfaphenazole (−0.715), pheniramine (−0.715), eldeline (−0.708), atropine methonitrate (−0.699), digitoxigenin (−0.679), fludroxycortide (−0.678), diflorasone (−0.669), tremorine (−0.664), 3-acetamidocoumarin (−0.66), benzocaine (−0.659), mevalolactone (−0.654), harmol (−0.643), sparteine (−0.643), digoxigenin (−0.636), tacrolimus (−0.634), lomustine (−0.628), fipexide (−0.614), lidoflazine (−0.614), alfaxalone (−0.611), suramin sodium (−0.61), chenodeoxycholic acid (−0.61), meticrane (−0.608), etidronic acid (−0.605), oxymetazoline (−0.604), amiprilose (−0.6), gibberellic acid (−0.597), helveticoside (−0.594), lanatoside C (−0.594), chloramphenicol (−0.589), cefotiam (−0.589), diphemanil metilsulfate (−0.589), guanethidine (−0.586), fursultiamine (−0.583), pivmecillinam (−0.574), letrozole (−0.573), memantine (−0.571), allantoin (−0.57), olivem 450 (−0.57), etacrynic acid (−0.569), raubasine (−0.566), 16-phenyltetranorprostaglandin E2 (−0.563), etomidate (−0.563), moracizine (−0.561), lidocaine (−0.56), ciclosporin (−0.556), fluticasone (−0.555), ofloxacin (−0.55), Dimethyl caffeic acid (−0.55), droperidol (−0.55), piromidic acid (−0.549), lisinopril (−0.549), atovaquone (−0.548), levodopa (−0.544), ajmaline (−0.541), velnacrine (−0.535), doxorubicin (−0.534), acebutolol (−0.534), tocamide (−0.534), sulpiride (−0.534), furaltadone (−0.533), pentamidine (−0.532), Citrus Extract (−0.53), iobenguane (−0.53), guaifenesin (−0.529), oleandomycin (−0.528), dizocilpine (−0.528), bezafibrate (−0.528), flumetasone (−0.526), nitrofural (−0.523), methyldopate (−0.522), stachydrine (−0.522), doxepin (−0.52), Pyrogallol (−0.52), amantadine (−0.52), midodrine (−0.52), biperiden (−0.519), monensin (−0.519), clebopride (−0.518), 16,16-dimethylprostaglandin E2 (−0.516), eucatropine (−0.516), niridazole (−0.516), tridihexethyl (−0.515), harpagoside (−0.513), palmatine (−0.512), Norcamphor (−0.51), dicloxacillin (−0.509), pronetalol (−0.508), triamcinolone (−0.506), gemfibrozil (−0.506), ronidazole (−0.505), deptropine (−0.505), raloxifene (−0.503), sulfamethoxazole (−0.503), 6-bromoindirubin-3′-oxime (−0.503), fusidic acid (−0.502), Raspberry ketone (−0.47), Omega-pentadecalactone (−0.47), Enoxolone (−0.40), Usnic acid (−0.38), Tartaric acid (−0.35), Resveratrol (−0.35), Peppermint oil (−0.33), Vanillin (−0.32), Rosmarinic acid (−0.30), Silymarin (−0.27), emulphor (−0.27), neem oil (−0.23), Caffeine (−0.22), Taurine (−0.20), D-Carvone (−0.18), Cis-Jasmone (−0.17), Rice Bran Oil (−0.07), Chrysin (−0.52), Thymol (−0.20), Myricetin (−0.29) and Apigenin (−0.50).

A variety of natural extracts (e.g. Vitamin A, B1, and B12) and flavonoids (e.g., fisetin, quercetin, vitexin, chrysin, narigenin, and harmin) demonstrated high negative connectivity to the rhinovirus infection biomarker profile. Several of the identified compounds possess anti-inflammatory effects in the rhinovirus infection mediated inflammation model, including kaempferol, luteolin, quercetin, genistein, troxerutin, daidzein, oxymetazoline, pyrrolidinedithiocarbamate ammonium, 7-hydroxyflavone, myricetin, eriodictyol, diosmetin, naringenin, chrysin, apigenin, 4′,7-dihydroxyflavone, scutellarein, all-transretinoic acid, 3-hydroxyflavone, fustin, 3-methylquercetin and fisetin.

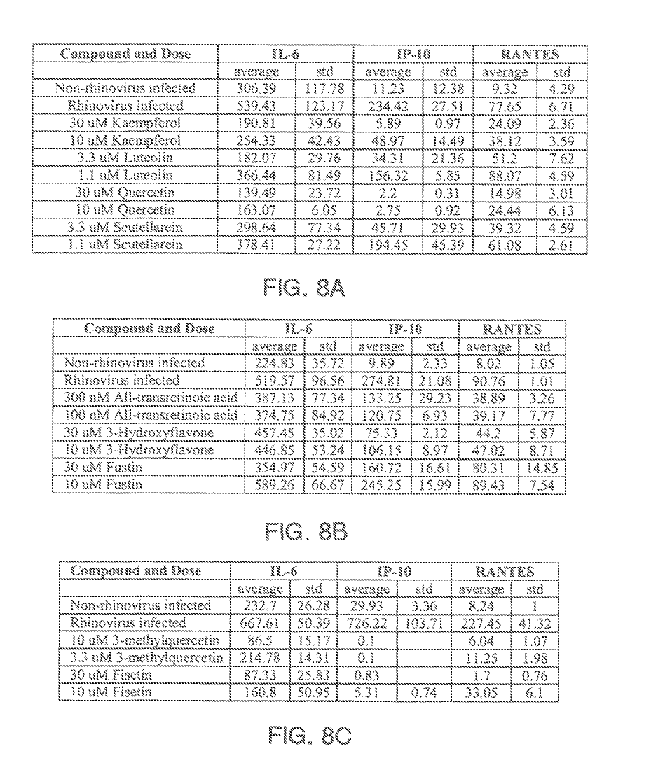

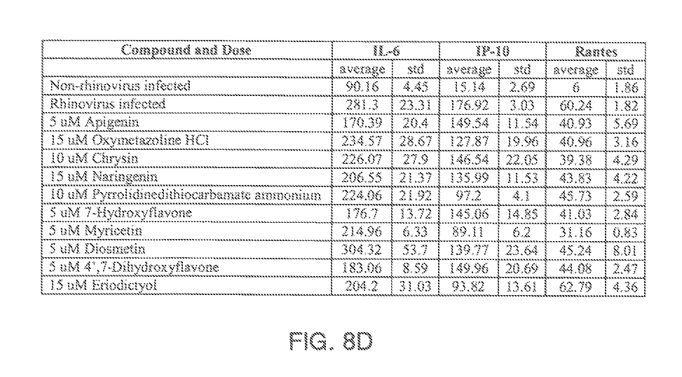

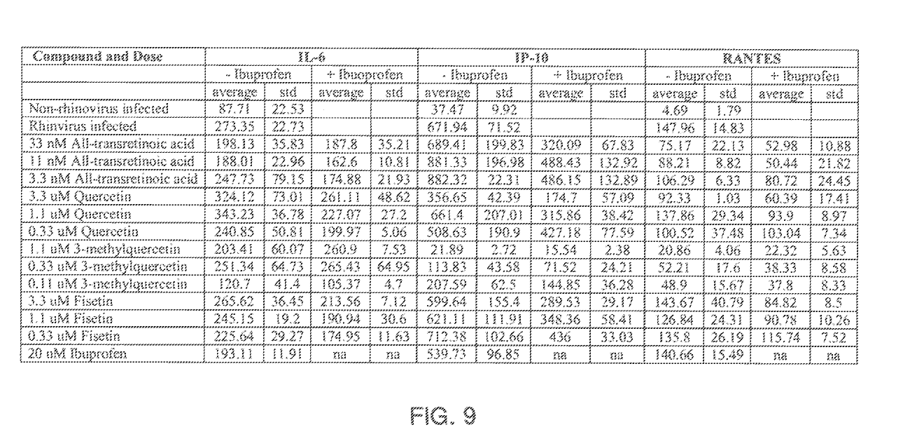

The effect of various identified compounds on rhinovirus infection-induced inflammatory markers (IL-6, IP-10, and RANTES) in the human bronchial epithelia cell line, BEAS-2B, was evaluated. Kaempferol, luteolin, quercetin, scutellarein, all-transretinoic acid, 3-hydroxyflavone, fustin, 3-methylquercetin, fisetin, apigenin, oxymetazoline HCl, chrysin, naringenin, pyrrolidinedithiocarbamate ammonium, 7-hydroxyflavone, myricetin, diosmetin, 4′,7-dihydroxyflavone, or eriodictyol was added to cell culture media at various doses at the time of RV-16 infection of BEAS-2B cells. IL-6, IP-10 and RANTES protein levels were measured three days post-infection. Cytotoxicity of the compounds was determined by the uptake of vital dye. Rhinovirus infected BEAS-2B cells not exposed to the compounds served as a positive control, achieving maximum cytokine production. BEAS-2B cells not exposed to rhinovirus or the compounds served as a negative control. Exemplary results are set forth in FIGS. 8A-8D. A vast majority of the tested compounds reduced rhinovirus infection-induced inflammatory markers in infected cells. The study was repeated for a subset of compounds, and ibuprofen (20 M) was added to the cell media with the compounds at the time of infection. IL-6, IP-10 and RANTES protein levels were measured two days post-infection. The results are provided in FIG. 9. In a majority of instances, addition of ibuprofen further reduced levels of biomarkers associated with rhinovirus infection.

This example describes the identification of excipients suitable for maintaining or improving respiratory health using the gene expression profile and methods of the invention.

### Example 3

A composition comprising an excipient identified as described herein is prepared as follows:

| Example | | Analgesic/Anti- | Antitus- | Decon- | Antihis- |
|---|---|---|---|---|---|
| Formu- | Excipient | inflammatory/ | sive | gestant | tamine |
| lation | (M) | Antipyretic (M) | (M) | (M) | (M) |
| 1 | 1-10 | 30 | 0.02 | 0.001 | 0.5 |
| 2 | 1-10 | 30 | 0.02 | 0.001 | x |
| 3 | 1-10 | 30 | 0.02 | 2 | 0.5 |
| 4 | 1-10 | 30 | 0.02 | 2 | x |
| 5 | 1-10 | 25 | 0.02 | 0.001 | 0.5 |
| 6 | 1-10 | 25 | 0.02 | 0.001 | x |
| 7 | 1-10 | 25 | 0.02 | 2 | 0.5 |
| 8 | 1-10 | 25 | 0.02 | 2 | x |

Suitable excipients, analgesics/anti-inflammatories/antipyretics, antitussives, decongestants, and antihistamines are described herein. Various exemplary formulations of the invention comprise ibuprofen (formations 1-4) or acetaminophen (formulations 5-8), dextromethorphan, phenylephrine (formations 1, 2, 5, and 6) or pseudoephedrine (formations 3, 4, 7, and 8), dexamethasone, and/or doxylamine. Alternatively, the decongestant is oxymetazoline (1 M). In various embodiments, the excipient is a flavonoid and/or anti-oxidant, such as quercetin (2 M), neohesperidin (3 M), propyl gallate (2 M), quercetin+neohesperidin+propyl gallate, naringenin (8 M), epicatechin 3-gallate (3 M), epigallocatechin (3 M), and/or epigallocatechin 3-gallate (2 M).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for evaluating the activity of an agent for treating rhinovirus infection or a symptom thereof, the method comprising:
a. administering an agent to an animal infected with rhinovirus, and
b. measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample from the animal to produce a gene expression profile,
wherein a gene expression profile that differs from a reference gene expression profile indicates that the agent treats rhinovirus infection or a symptom thereof.

2. The method of claim 1, wherein step (b) comprises measuring expression of five or more of the genes.

3. The method of claim 1, wherein step (b) comprises measuring mRNA encoded by one or more of the genes.

4. The method of claim 3, wherein mRNA is measured by hybridization to an array comprising oligonucleotides specific for mRNA encoded by one or more of the genes.

5. The method of claim 1, wherein step (b) comprises reverse transcribing mRNA encoded by one or more of the genes and measuring corresponding cDNA.

6. The method of claim 1, wherein step (b) comprises measuring protein encoded by one or more of the genes.

7. The method of claim 1, wherein the reference gene expression profile is a reference gene expression profile of infection, and an increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or a decrease in expression of FAS, PLSCR1, CLEC2B, BATF3, HAS2, MX1, SP110, GBP1, IFIT3, IFIT1, CXCL9, CXCL10, and/or CXCL11 in the gene expression profile compared to the reference gene expression profile of infection indicates that the agent treats rhinovirus infection or a symptom thereof.

8. A method of treating rhinovirus infection or a symptom thereof, the method comprising:
a. administering to a human patient an agent according to a therapeutic regimen for treating rhinovirus infection or a symptom thereof,
b. measuring expression of (i) one or more genes selected from the group consisting of CRY2, B3GAT3, C10ORF95, and BATF3, and (ii) one or more genes selected from the group consisting of RNFT2, BTG4, PSD3, CAPN9, SULT1E1, HEY1, LRRC36, RAB3B, ALDH3B1, FAM134B, FAS, PLSCR1, CLEC2B, HAS2, MX1, SP110, GBP1, IFIT3, IFIT1, CXCL9, CXCL10, and CXCL11, from at least one biological sample from the patient to generate a gene expression profile;
c. comparing the gene expression profile from the patient to a reference gene expression profile; and
d. generating a new therapeutic regimen for the patient to treat rhinovirus infection or a symptom thereof if the gene expression profile (i) differs from a reference gene expression profile of non-infection or (ii) does not significantly differ from a reference gene expression profile of infection.

9. The method of claim 8, wherein step (c) comprises comparing the gene expression profile to a reference gene expression profile of non-infection, and step (d) comprises generating a new therapeutic regimen for the patient if the gene expression profile comprises a decrease in expression of RNFT2, CRY2, C10ORF95, BTG4, PSD3, CAPN9, SULT1E1, HEY1, LRRC36, RAB3B, ALDH3B1, FAM134B, and/or B3GAT3, and/or an increase in expression of FAS, PLSCR1, CLEC2B, BATF3, HAS2, MX1, SP110, GBP1, IFIT3, IFIT1, CXCL9, CXCL10, and/or CXCL11, compared to the reference gene expression profile of non-infection.

10. The method of claim 8, wherein step (c) comprises comparing the gene expression profile to a reference gene expression profile of infection, and step (d) comprises generating a new therapeutic regimen for the patient if the gene expression profile comprises an insignificant increase in expression of RNFT2, CRY2, C10ORF95, BTG4, PSD3, CAPN9, SULT1E1, HEY1, LRRC36, RAB3B, ALDH3B1, FAM134B, and/or B3GAT3, and/or an insignificant decrease in expression of FAS, PLSCR1, CLEC2B, BATF3, HAS2, MX1, SP110, GBP1, IFIT3, IFIT1, CXCL9, CXCL10, and/or CXCL11, compared to the reference gene expression profile of infection.

11. The method of claim 8, wherein step (b) further comprises measuring expression of one or more additional genes selected from the group consisting of ACCN2, ADCY2, ADH6, ALDH5A1, BCL2L14, BTBD3, C11ORF16, C19ORF66, C5ORF4, C5ORF42, C7ORF63, C9ORF116, CASC1, CCDC81, CDCl₄A, CX3CL1, DDX60, DNAH6, ETV7, GCH1, GOLGA2B, GZMB, HERC6, HHLA2, IF144, IF144L, IFIH1, IFIT2, IFIT5, IFITM1, IQCH, ISG15, LRP2BP, LRRC23, LRRC50, MX2, NDRG1, OAS2, OSMR, PACRG, PCOTH, PCSK5, PMAIP1, PML, PTGFR, SLC16A1, SLC25A28, SOCS1, SPAG8, STARD5, TBC1D8, TRIM22, TRIM3, TSPAN8, USP2, and XAF1.

12. The method of claim 11, wherein step (b) further comprises measuring expression of 10 or more of the additional genes.

13. A composition comprising (i) a therapeutic agent selected from the group consisting of an antihistamine, an antiitussive, a decongestant, an expectorant, and combinations thereof, and (ii) an excipient selected from the group consisting of olivem 450, usnic acid, silymarin, gum rosin, trideceth-10, and combinations thereof.

14. The composition of claim 13, wherein the excipient is selected from the group consisting of olivem 450, silymarin, gum rosin, and trideceth-10.

15. The composition of claim 13, wherein the antihistamine is selected from the group consisting of brompheniramine, dexbrompheniramine, chlorpheniramine, cetirizine, levocetirizine, diphenhydramine, doxylamine, fexofenadine, loratadine, desloratadine, and combinations thereof.

16. The composition of claim 13, wherein the antiitussive is selected from the group consisting of dextromethorphan, menthol, codeine, chlophedianol, levodropropizine, and combinations thereof.

17. The composition of claim 13, wherein the decongestant is selected from the group consisting of pseudoephedrine, phenylephrine, ephedrine, and combinations thereof.

18. The composition of claim 13, wherein the expectorant is selected from the group consisting of guaifenesin, ambroxol, bromhexine, and combinations thereof.

19. The composition of claim 13 formulated for oral administration to a human.

20. The composition of claim 13 formulated for nasal administration to a human.
